(19) United States
(12) Patent Application Publication
(10) Pub. No.: US 2026/0354780 A1
(43) Pub. Date: Nov. 12, 2026

(54) GENERATION OF SYNTHETIC RADIOLOGICAL IMAGES

(71) Applicant: BAYER AKTIENGESELLSCHAFT, Leverkusen (DE)

(72) Inventors: Matthias LENGA, Leverkusen (DE); Ivo Matteo BALTRUSCHAT, Leverkusen (DE); Felix Karl KREIS, Leverkusen (DE)

(21) Appl. No.: 19/107,228

(22) PCT Filed: Aug. 23, 2023
(86) PCT No.: PCT/EP2023/073103

§ 371 Date: Feb. 27, 2025

(30) Foreign Application Priority Data
Aug. 30, 2022 (EP) 22192907.8
Mar. 1, 2023 (EP) 23159289.0

(51) Int. Cl. A61B 6/00; A61K 49/10; G06T 7/00

(52) U.S. Cl. CPC A61B 6/481; A61B 6/5205; A61K 49/108; G06T 7/0012; G06T 2207/10116; G06T 2207/20081; G06T 2207/20084

(57) ABSTRACT

The present invention relates to the technical field of radiology, and in particular to assisting radiologists in radiological examinations using artificial intelligence methods. The present invention relates to training a machine learning model and using the trained model to predict a synthetic representation of an examination area of an object to be examined.

### TECHNICAL FIELD

The present disclosure relates to the technical field of radiology, in particular that of assisting radiologists in radiological examinations using artificial intelligence methods. The present invention is concerned with the training of a machine-learning model and the utilization of the trained model for predicting a synthetic representation of an examination region of an examination object.

### INTRODUCTION

Tracking processes over time within the body of a human or animal using imaging methods plays an important role, inter alia, in the diagnosis and/or therapy of diseases.

An example that may be mentioned is the detection and differential diagnosis of focal liver lesions by means of dynamic contrast-enhanced magnetic resonance imaging (MRI) using a hepatobiliary contrast agent.

A hepatobiliary contrast agent such as Primovist® can be used for the detection of tumors in the liver. Blood is supplied to healthy liver tissue primarily via the portal vein (vena portae), whereas most primary tumors are supplied by the liver artery (arteria hepatica). After intravenous injection of a bolus of contrast agent, it is accordingly possible to observe a time delay between the signal increase in the healthy liver parenchyma and in the tumor.

Besides malignant tumors, what are commonly found in the liver are benign lesions such as cysts, hemangiomas and focal nodular hyperplasias (FNH). A proper planning of therapy requires that these be differentiated from the malignant tumors. Primovist® can be used for the identification of benign and malignant focal liver lesions. By means of T1-weighted MRI, it provides information about the character of said lesions. Differentiation is achieved by utilizing the difference in blood supply to liver and tumor and the time profile of contrast enhancement.

In the case of the contrast enhancement achieved by Primovist® during the wash-in phase, what are observed are typical perfusion patterns which provide information for the characterization of the lesions. Depicting the vascularization helps to characterize the lesion types and to determine the spatial relationship between tumor and blood vessels.

In the case of T1-weighted MRI images, Primovist® leads, 10-20 minutes after the injection (in the hepatobiliary phase), to a distinct signal enhancement in the healthy liver parenchyma, whereas lesions containing no hepatocytes or only a few hepatocytes, for example metastases or moderately to poorly differentiated hepatocellular carcinomas (HCCs), appear as darker regions.

Tracking the spread of the contrast agent over time thus provides a good way of detecting and differentially diagnosing focal liver lesions; however, the examination extends over a comparatively long period of time. Over this period of time, movements by the patient should be largely avoided in order to minimize movement artifacts in the MRI images. The lengthy restriction of movement can be unpleasant for a patient.

In the laid-open application WO2021/052896A1, it is proposed that one or more MRI images during the hepatobiliary phase not be generated by measurement, but that they be calculated (predicted) on the basis of MRI images from one or more preceding phases in order to shorten the time spent by the patient in the MRI scanner.

The approach described in the laid-open application WO2021/052896A1 involves training a machine-learning model to use MRI images of an examination region before and/or immediately after the administration of a contrast agent as a basis to predict an MRI image of the examination region at a later time.

It has been found that the radiological images predicted in this way may contain artifacts. Either fine structures may not be correctly reproduced, or regions of the synthetic radiological image may contain structures not found in the represented tissue (see for example: K. Schwarz et al.: On the Frequency Bias of Generative Models, https://doi.org/10.48550/arXiv.2111.02447).

### SUMMARY

This and other problems are addressed by the present disclosure.

The present disclosure provides in a first aspect a method for training a machine-learning model. The training method comprises:
receiving and/or providing training data, the training data comprising a set of input data and target data for each examination object of a multiplicity of examination objects,
   each set comprising at least one input representation of an examination region of the examination object as input data and a target representation of the examination region of the examination object and a transformed target representation as target data,
   the at least one input representation representing the examination region in a first period of time before and/or after the administration of a contrast agent and the target representation representing the examination region in a second period of time after the administration of the contrast agent,
   the transformed target representation representing at least part of the examination region of the examination object
      in frequency space, if the target representation represents the examination region of the examination object in real space, or
      in real space, if the target representation represents the examination region of the examination object in frequency space,
training a machine-learning model, the machine-learning model being configured to generate on the basis of at least one input representation of an examination region of an examination object and model parameters a synthetic representation of the examination region of the examination object,
wherein the training comprises for each examination object of the multiplicity of examination objects:
   feeding the at least one input representation to the machine-learning model,
   receiving a synthetic representation of the examination region of the examination object from the machine-learning model,
   generating and/or receiving a transformed synthetic representation on the basis of the synthetic representation and/or in relation to the synthetic representation, the transformed synthetic representation representing at least part of the examination region of the examination object
      in frequency space, if the synthetic representation represents the examination region of the examination object in real space, or
      in real space, if the synthetic representation represents the examination region of the examination object in frequency space,
   quantifying the differences i) between at least part of the synthetic representation and at least part of the target representation and ii) between at least part of the transformed synthetic representation and at least part of the transformed target representation by means of a loss function,
   reducing the differences by modifying model parameters,
outputting and/or storing the trained machine-learning model and/or the model parameters and/or transmitting the trained machine-learning model and/or the model parameters to a separate computer system and/or using the trained machine-learning model for generation of a synthetic radiological image of the examination region of a new examination object.

The present disclosure further provides a computer-implemented method (prediction method) for generating a synthetic radiological image with the aid of the trained machine-learning model. The prediction method comprises:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) at least one input representation of the examination region of the examination object, ii) a target representation of the examination region of the examination object and iii) a transformed target representation,
      the at least one input representation representing the examination region of the respective examination object in a first period of time before or after administration of a contrast agent,
      the target representation representing the examination region of the respective examination object in a second period of time after the administration of the contrast agent,
      the transformed target representation representing at least part of the examination region of the respective examination object in frequency space, if the target representation represents the examination region of the respective examination object in real space, or in real space, if the target representation represents the examination region of the respective examination object in frequency space,
   the training of the machine-learning model comprising reducing differences between i) at least part of the synthetic representation and at least part of the target representation and ii) between at least part of a transformed synthetic representation and at least part of the transformed target representation,
receiving at least one input representation of the examination region of a new examination object, the at least one input representation of the examination region of the new examination object representing the examination region in a first period of time before and/or after administration of a contrast agent,
inputting the at least one input representation of the examination region of the new examination object into the trained machine-learning model,
receiving a synthetic representation of the examination region of the new examination object from the machine-learning model,
outputting and/or storing the synthetic representation of the examination region of the new examination object and/or transmitting the synthetic representation of the examination region of the new examination object to a separate computer system.

The present disclosure further provides a computer system comprising
a receiving unit,
a control and calculation unit and
an output unit,
   wherein the control and calculation unit is configured to provide a trained machine-learning model,
      the trained machine-learning model having been trained by means of training data to generate on the basis of at least one input representation of an examination region of an examination object a synthetic representation of the examination region of the examination object,
      the training data comprising for each examination object of a multiplicity of examination objects i) at least one input representation of the examination region of the examination object, ii) a target representation of the examination region of the examination object and iii) a transformed target representation,
         the at least one input representation representing the examination region of the respective examination object in a first period of time before or after administration of a contrast agent,
         the target representation representing the examination region of the respective examination object in a second period of time after the administration of the contrast agent,
         the transformed target representation representing at least part of the examination region of the respective examination object in frequency space, if the target representation represents the examination region of the respective examination object in real space, or in real space, if the target representation represents the examination region of the respective examination object in frequency space,
      the training of the machine-learning model comprising reducing differences between i) at least part of the synthetic representation and at least part of the target representation and ii) between at least part of a transformed synthetic representation and at least part of the transformed target representation,
   wherein the control and calculation unit is configured to cause the receiving unit to receive at least one input representation of an examination region of a new examination object, the at least one input representation of the examination region of the new examination object representing the examination region in a first period of time before and/or after administration of a contrast agent,
   wherein the control and calculation unit is configured to input the at least one input representation of the examination region of the new examination object into a trained machine-learning model,
   wherein the control and calculation unit is configured to receive from the machine-learning model a synthetic representation of the examination region of the new examination object,
   wherein the control and calculation unit is configured to cause the output unit to output the synthetic representation of the examination region of the new examination object and/or to store it and/or to transmit it to a separate computer system.

The present disclosure further provides a computer program product comprising a computer program that can be loaded into a working memory of a computer system, where it causes the computer system to execute the following steps:
providing a trained machine-learning model,
the trained machine-learning model having been trained by means of training data to generate on the basis of at least one input representation of an examination region of an examination object a synthetic representation of the examination region of the examination object,
   the training data comprising for each examination object of a multiplicity of examination objects i) at least one input representation of the examination region of the examination object, ii) a target representation of the examination region of the examination object and iii) a transformed target representation,
      the at least one input representation representing the examination region of the respective examination object in a first period of time before or after administration of a contrast agent,
      the target representation representing the examination region of the respective examination object in a second period of time after the administration of the contrast agent,
      the transformed target representation representing at least part of the examination region of the respective examination object in frequency space, if the target representation represents the examination region of the respective examination object in real space, or in real space, if the target representation represents the examination region of the respective examination object in frequency space,
   the training of the machine-learning model comprising reducing differences between i) at least part of the synthetic representation and at least part of the target representation and ii) between at least part of a transformed synthetic representation and at least part of the transformed target representation,
receiving at least one input representation of the examination region of a new examination object, the at least one input representation of the examination region of the new examination object representing the examination region in a first period of time before and/or after administration of a contrast agent,
inputting the at least one input representation of the examination region of the new examination object into the trained machine-learning model,
receiving a synthetic representation of the examination region of the new examination object from the machine-learning model,
outputting and/or storing the synthetic representation of the examination region of the new examination object and/or transmitting the synthetic representation of the examination region of the new examination object to a separate computer system.

The present disclosure further provides for the use of a contrast agent in a radiological examination method, the radiological examination method comprising:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) at least one input representation of the examination region of the examination object, ii) a target representation of the examination region of the examination object and iii) a transformed target representation,
      the at least one input representation representing the examination region of the respective examination object in a first period of time before or after administration of the contrast agent,
      the target representation representing the examination region of the respective examination object in a second period of time after the administration of the contrast agent,
      the transformed target representation representing at least part of the examination region of the respective examination object in frequency space, if the target representation represents the examination region of the respective examination object in real space, or in real space, if the target representation represents the examination region of the respective examination object in frequency space,
   the training of the machine-learning model comprising reducing differences between i) at least part of the synthetic representation and at least part of the target representation and ii) between at least part of a transformed synthetic representation and at least part of the transformed target representation,
receiving at least one input representation of the examination region of a new examination object, the at least one input representation of the examination region of the new examination object representing the examination region in a first period of time before and/or after administration of a contrast agent,
inputting the at least one input representation of the examination region of the new examination object into the trained machine-learning model,
receiving a synthetic representation of the examination region of the new examination object from the machine-learning model,
outputting and/or storing the synthetic representation of the examination region of the new examination object and/or transmitting the synthetic representation of the examination region of the new examination object to a separate computer system.

The present invention further provides a contrast agent for use in a radiological examination method, the radiological examination method comprising:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) at least one input representation of the examination region of the examination object, ii) a target representation of the examination region of the examination object and iii) a transformed target representation,
      the at least one input representation representing the examination region of the respective examination object in a first period of time before or after administration of the contrast agent,
      the target representation representing the examination region of the respective examination object in a second period of time after the administration of the contrast agent,
      the transformed target representation representing at least part of the examination region of the respective examination object in frequency space, if the target representation represents the examination region of the respective examination object in real space, or in real space, if the target representation represents the examination region of the respective examination object in frequency space,
   the training of the machine-learning model comprising reducing differences between i) at least part of the synthetic representation and at least part of the target representation and ii) between at least part of a transformed synthetic representation and at least part of the transformed target representation,
receiving at least one input representation of the examination region of a new examination object, the at least one input representation of the examination region of the new examination object representing the examination region in a first period of time before and/or after administration of a contrast agent,
inputting the at least one input representation of the examination region of the new examination object into the trained machine-learning model,
receiving a synthetic representation of the examination region of the new examination object from the machine-learning model,
outputting and/or storing the synthetic representation of the examination region of the new examination object and/or transmitting the synthetic representation of the examination region of the new examination object to a separate computer system.

The present disclosure further provides a kit comprising a contrast agent and a computer program product comprising a computer program that can be loaded into a working memory of a computer system, where it causes the computer system to execute the following steps:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) at least one input representation of the examination region of the examination object, ii) a target representation of the examination region of the examination object and iii) a transformed target representation,
      the at least one input representation representing the examination region of the respective examination object in a first period of time before or after administration of the contrast agent,
      the target representation representing the examination region of the respective examination object in a second period of time after the administration of the contrast agent,
      the transformed target representation representing at least part of the examination region of the respective examination object in frequency space, if the target representation represents the examination region of the respective examination object in real space, or in real space, if the target representation represents the examination region of the respective examination object in frequency space,
   the training of the machine-learning model comprising reducing differences between i) at least part of the synthetic representation and at least part of the target representation and ii) between at least part of a transformed synthetic representation and at least part of the transformed target representation,
receiving at least one input representation of the examination region of a new examination object, the at least one input representation of the examination region of the new examination object representing the examination region in a first period of time before and/or after administration of a contrast agent,
inputting the at least one input representation of the examination region of the new examination object into the trained machine-learning model,
receiving a synthetic representation of the examination region of the new examination object from the machine-learning model,
outputting and/or storing the synthetic representation of the examination region of the new examination object and/or transmitting the synthetic representation of the examination region of the new examination object to a separate computer system.

Further subjects and embodiments can be found in the following description, the claims and the drawings.

### BRIEF DESCRIPTION OF THE DRAWINGS

FIG. 1 shows by way of example and in schematic form the process of training the machine-learning model.

FIG. 2 shows by way of example and in schematic form a use of a trained machine-learning model for prediction.

FIG. 3 shows by way of example and in schematic form a further embodiment of the training of the machine-learning model.

FIG. 4 shows by way of example and in schematic form a further embodiment of the training of the machine-learning model.

FIG. 5 shows by way of example and in schematic form a further embodiment of the training of the machine-learning model.

FIG. 6 shows by way of example and in schematic form a further embodiment of the training of the machine-learning model.

FIG. 7 shows by way of example and in schematic form a further embodiment of the training of the machine-learning model.

FIG. 8 shows by way of example and in schematic form a further embodiment of the training of the machine-learning model.

FIG. 9 shows by way of example and in schematic form a further use of a trained machine-learning model for prediction.

FIG. 10 shows by way of example and in schematic form a computer system according to the present disclosure.

FIG. 11 shows by way of example and in schematic form a further embodiment of the computer system according to the present disclosure.

FIG. 12 shows schematically in the form of a flow chart one embodiment of the method for training a machine-learning model.

FIG. 13 shows schematically in the form of a flow chart one embodiment of the method for generating a synthetic representation of an examination region of an examination object with the aid of the trained machine-learning model.

### DETAILED DESCRIPTION

The invention will be more particularly elucidated below without distinguishing between the subjects of the invention (training method, prediction method, computer system, computer program product, use, contrast agent for use, kit). Rather, the following remarks are intended to apply mutatis mutandis to all subjects of the invention (training method, prediction method, computer system, computer program product, use, contrast agent for use, kit), irrespective of the context in which the remarks are made.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

With the aid of the present invention, representations of an examination region of an examination object can be predicted. Such a predicted representation is also referred to as a synthetic representation in this disclosure.

The “examination object” is normally a living being, preferably a mammal, most preferably a human.

The “examination region” is part of the examination object, for example an organ or part of an organ, such as the liver, brain, heart, kidney, lung, stomach, intestines, pancreas, thyroid gland, prostate, breast or part of the aforementioned organs, or multiple organs or another part of the body.

The “examination region” is part of the examination object, for example an organ or part of an organ, such as the liver, brain, heart, kidney, lung, stomach, intestines, pancreas, thyroid gland, prostate, breast or part of the aforementioned organs, or multiple organs or another part of the body.

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

A “representation of the examination region” is normally the result of a radiological examination.

“Radiology” is the branch of medicine that is concerned with the use of predominantly electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present invention thus encompasses in particular the following examination methods: computed tomography, magnetic resonance imaging, sonography.

In a preferred embodiment of the present invention, the radiological examination is a magnetic resonance imaging examination or computed tomography examination. Most preferably, the radiological examination is a magnetic resonance imaging examination.

Magnetic resonance imaging, MRI for short, is an imaging method that is used especially in medical diagnostics for depicting structure and function of tissues and organs in the human or animal body.

In MRI, the magnetic moments of protons in an examination object are aligned in a basic magnetic field, with the result that there is a macroscopic magnetization along a longitudinal direction. This is then deflected from the resting position by irradiation with high-frequency (HF) pulses (excitation). The return of the excited states to the resting position (relaxation), or magnetization dynamics, is then detected as relaxation signals by means of one or more HF receiver coils.

For spatial encoding, rapidly switched magnetic gradient fields are superimposed on the basic magnetic field. The captured relaxation signals, or detected MRT data, are initially in the form of raw data in frequency space (so-called k-space data), and can be transformed by subsequent inverse Fourier transform into real space (image space).

A representation of the examination region in the context of the present disclosure may be an MRI image, a CT image, an ultrasound image or the like, or the representation of the examination region may be generated from one or more MRI images, CT images, ultrasound images or the like.

In radiological examinations, contrast agents are commonly used for contrast enhancement.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (e.g. iron oxide nanoparticles, superparamagnetic iron-platinum particles (SIPPs)) or paramagnetic substances (e.g. gadolinium chelates, manganese chelates) are usually used as contrast agents. In the case of sonography, liquids containing gas-filled microbubbles are usually administered intravenously. Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M.R. Nouh et al.: Radiographic and magnetic resonances contrast agents: Essentials and tips for safe practices, World J Radiol. 2017 Sep. 28; 9(9): 339-349; L.C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

MRI contrast agents exert their effect by altering the relaxation times of the structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), and gadoxetic acid (Primovist®/Eovist®).

The radiological examination method may be a magnetic resonance imaging examination method and the contrast agent an MRI contrast agent. This means that the at least one input representation may be at least one MRI image and the synthetic representation a synthetic MRI image.

The radiological examination method may be a computed tomography examination method and the contrast agent a CT contrast agent. This means that the at least one input representation may be at least one CT image and the synthetic representation a synthetic CT image.

It is also possible that the at least one input representation comprises at least one MRI image and at least one CT image.

It is also possible that the radiological examination method is a computed tomography examination method and the contrast agent an MRI contrast agent.

In a preferred embodiment, the contrast agent is an MRI contrast agent (irrespective of whether it is used in a magnetic resonance tomography examination method or in a computed tomography method).

The MRI contrast agent may be an extracellular contrast agent. Extracellular contrast agents refer to low-molecular-weight, water-soluble compounds that, after intravenous administration, are distributed to the blood vessels and the interstitial spaces. After a certain, comparatively short period of circulation in the bloodstream, they are excreted via the kidneys. Extracellular MRI contrast agents include, for example, the gadolinium chelates gadobutrol (Gadovist®), gadoteridol (Prohance®), gadoteric acid (Dotarem®), gadopentetic acid (Magnevist®) and gadodiamide (Omnican®).

The MRI contrast agent may be an intracellular contrast agent. Intracellular contrast agents are taken up into the cells of tissues to a certain extent and subsequently excreted. Intracellular MRI contrast agents based on gadoxetic acid are distinguished for example in that they undergo a degree of specific uptake by liver cells (hepatocytes), accumulate in the functional tissue (parenchyma) and enhance contrasts in healthy liver tissue, before being subsequently excreted via the gallbladder into the feces. Examples of such contrast agents based on gadoxetic acid are described in U.S. Pat. No. 6,039,931A; they are commercially available for example under the trade names Primovist® and Eovist®. A further MRI contrast agent having a lower uptake into the hepatocytes is gadobenate dimeglumine (Multihance®).

Gadoxetate disodium (GD, Primovist®) belongs to the group of intracellular contrast agents. It is authorized for use in MRI of the liver for detecting and characterizing lesions in patients with known or suspected focal liver disease. With its lipophilic ethoxybenzyl unit, GD exhibits two-phase spreading: spreading at first in the intravascular and interstitial space after bolus injection, followed by selective uptake by hepatocytes. GD is excreted from the body unaltered via the kidneys and the hepatobiliary route (50:50 dual mechanism of excretion) in about the same amounts. Because of its selective accumulation in healthy liver tissue, GD is also referred to as a hepatobiliary contrast agent.

GD is approved at a dose of 0.1 ml/kg body weight (BW) (0.025 mmol/kg BW Gd). The recommended administration of GD comprises an undiluted intravenous bolus injection at a flow rate of about 2 ml/second, followed by flushing of the i.v. cannula with a physiological saline solution. A standard protocol for liver imaging using GD consists of multiple planning and pre-contrast sequences. After i.v. bolus injection of the contrast agent, dynamic images are usually acquired during the arterial phase (about 30 seconds after the injection, p.i.), portal venous phase (about 60 seconds p.i.) and transitional phase (about 2-5 minutes p.i.). Typically, the transitional phase already shows a certain rise in the liver signal intensity owing to the incipient uptake of the agent into hepatocytes. Additional T2-weighted and diffusion-weighted (DWI) images can be generated after the dynamic phase and before the late hepatobiliary phase.

In one embodiment, the contrast agent is gadoxetate disodium.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(+)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that includes a Gd³⁺ complex of a compound of the formula (I)

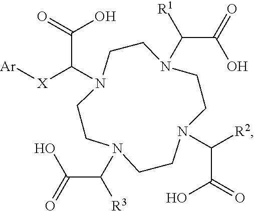

where
Ar is a group selected from

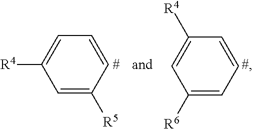

where _{#} is the linkage to X,
X is a group selected from
CH₂, (CH₂)₂, (CH₂)₃, (CH₂)₄ and *—(CH₂)₂O—CH₂—^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that includes a Gd³⁺ complex of a compound of the formula (II)

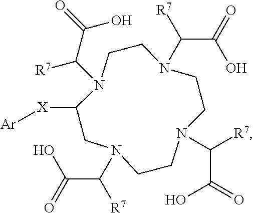

where
Ar is a group selected from

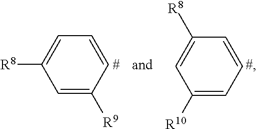

where _{#} is the linkage to X,
X is a group selected from CH₂, (CH₂)₂, (CH₂)₃, (CH₂)₄ and *—(CH₂)₂O—CH₂—, where * is the linkage to Ar and ^{#} is the linkage to the acetic acid residue,
R⁷ is a hydrogen atom or a group selected from C₁-C₃ alkyl, —CH₂OH,
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” denotes a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

A representation of the examination region in the context of the present disclosure may be a representation in real space (image space) or a representation in frequency space.

In a representation in real space, also referred to in this description as real-space depiction or real-space representation, the examination region is normally represented by a large number of image elements (pixels or voxels) that may for example be in a raster arrangement, in which case each image element represents a part of the examination region. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental frequencies. For example, the examination region may be represented by a sum of sine and cosine functions having different amplitudes, frequencies and phases. The amplitudes and phases may be plotted as a function of the frequencies, for example, in a two- or three-dimensional representation. Normally, the lowest frequency (origin) is placed in the center. The further away from this center, the higher the frequencies. Each frequency can be assigned an amplitude representing the frequency in the frequency-space depiction and a phase indicating the extent of the shift of the respective wave with respect to a sine or cosine wave.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https://see.stanford.edu/materials/lsoftaee261/book-fall-07.pdf.

With the aid of a trained machine-learning model, it is possible to generate on the basis of at least one representation of an examination region of an examination object a synthetic representation of the examination region of the examination object. The at least one representation, on the basis of which the (trained) machine-learning model generates the synthetic representation, is also referred to in this description as an input representation.

The synthetic representation normally represents the same examination region of the same examination object as the at least one input representation, on the generation of which the synthetic representation is based.

The at least one input representation represents the examination region of an examination object in a first period of time before and/or after administration of a contrast agent. The synthetic representation represents the examination region in a second period of time after the administration of the contrast agent.

The second period of time preferably follows the first period of time, i.e. it is preferably later than the first period of time. The second period of time may immediately follow the first period of time; however, it is also possible for there to be a time interval between the first period of time and the second period of time.

The at least one input representation may comprise, for example, a first input representation of the examination region representing the examination region before administration of a contrast agent and a second input representation representing the examination region after the administration of a contrast agent. The first input representation and the second input representation represent the examination region in a first period of time. The period of time begins at a first moment before the administration of the contrast agent and ends at a second moment after the administration of the contrast agent. The synthetic representation represents the examination region at a third moment in a second period of time after the administration of the contrast agent.

The at least one input representation may also comprise a first input representation of the examination region representing the examination region at a first moment in a period of time after administration of a contrast agent and a second input representation representing the examination region at a second moment in the first period of time after the administration of the contrast agent. The synthetic representation may represent the examination region at a third moment in a second period of time after the administration of the contrast agent.

It is also conceivable that the at least one input representation comprises more than two input representations of the examination region in a first period of time before and/or after the administration of a contrast agent, for example three or four or five or some other number. Each of the input representations may represent the examination region at a different moment before and/or after the administration of the contrast agent. However, it is also conceivable that two or more input representations represent the examination region at the same moment.

It is also conceivable that the at least one input representation is only one input representation of the examination region in a first period of time before or after the administration of a contrast agent.

It is also conceivable that the at least one input representation represents the examination region in a first period of time before and/or after a first administration of a contrast agent and the synthetic representation represents the examination region before and/or after a second administration of a contrast agent. The contrast agent of the first administration and the contrast agent of the second administration may be the same or different.

In one embodiment, a machine-learning model is trained to predict a synthetic representation of a liver or part of a liver of an examination object in the hepatobiliary phase of an MRI examination on the basis of at least one input representation, the at least one input representation representing the liver or part of the liver of the examination object at one or more earlier moments. The at least one input representation may represent the liver or part of the liver, for example, before the administration of a hepatobiliary contrast agent and/or in the arterial phase after the administration of the hepatobiliary contrast agent and/or in the portal venous phases after the administration of the hepatobiliary contrast agent and/or in the transitional phase after the administration of the hepatobiliary contrast agent.

After the intravenous administration of the hepatobiliary contrast agent in the form of a bolus into an arm vein, the contrast agent reaches the liver first via the arteries. These are depicted with contrast enhancement in the corresponding MRI images. The phase in which the liver arteries are depicted with contrast enhancement in MRI images is referred to as “arterial phase”.

Subsequently, the contrast agent reaches the liver via the liver veins. Whereas the contrast in the liver arteries is already decreasing, the contrast in the liver veins is reaching a maximum. The phase in which the liver veins are depicted with contrast enhancement in MRI images is referred to as “portal venous phase”. Said phase can already start during the arterial phase and overlap therewith.

The portal venous phase is followed by the “transitional phase”, in which the contrast in the liver arteries drops further and the contrast in the liver veins likewise drops. When using a hepatobiliary contrast agent, the contrast in the healthy liver cells gradually rises in the transitional phase.

The arterial phase, the portal venous phase and the transitional phase are also referred to collectively as “dynamic phase”.

10-20 minutes after its injection, a hepatobiliary contrast agent leads to a distinct signal enhancement in the healthy liver parenchyma. This phase is referred to as “hepatobiliary phase”. The contrast agent is eliminated only slowly from the liver cells; accordingly, the hepatobiliary phase can last for two hours and longer.

The stated phases are described in greater detail, for example, in the following publications: J. Magn. Reson. Imaging, 2012, 35(3): 492-511, doi:10.1002/jmri.22833; Clujul Medical, 2015, Vol. 88 No. 4: 438-448, DOI: 10.15386/cjmed-414; Journal of Hepatology, 2019, Vol. 71: 534-542, http://dx.doi.org/10.1016/j.jhep.2019.05.005).

Since a representation of the liver in the hepatobiliary phase is generated by means of a machine model in the form of a synthetic representation and is not measured, the length of time that has to be spent by an examination object in an MRI scanner is shorter. This also applies analogously to other examinations which extend over a period of time. The synthetic representations generated with the aid of the trained machine-learning model of the present disclosure are of higher quality than the synthetic representations generated by the method described in WO2021/052896A1. Moreover, the approach described herein makes it possible to specifically focus on defined frequency ranges when training the machine-learning model and thus control what information is to be depicted with particular accuracy by the synthetic representations.

A “machine-learning model” can be understood as a computer-implemented data processing architecture. The model is able to receive input data and to supply output data on the basis of said input data and model parameters. The model can learn a relationship between the input data and the output data by means of training. During training, model parameters can be adjusted so as to supply a desired output for a particular input.

During the training of such a model, the model is presented with training data from which it can learn. The trained machine-learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum. The modification of model parameters in order to reduce the differences can be done using an optimization method such as a gradient descent method.

The differences can be quantified with the aid of a loss function. A loss function of this kind can be used to calculate a loss value for a given pair of output data and target data. The goal of the training process can consist in altering (adjusting) the parameters of the machine-learning model so as to reduce the loss value for all pairs of the training data set to a (defined) minimum.

For example, if the output data and the target data are numbers, the loss function can be the absolute difference between these numbers. In this case, a high absolute loss value can mean that one or more model parameters need to be altered to a substantial degree.

For example, for output data in the form of vectors, difference metrics between vectors such as the mean squared error, a cosine distance, a norm of the difference vector such as a Euclidean distance, a Chebyshev distance, an Lp norm of a difference vector, a weighted norm or any other type of difference metric between two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

In the present case, the machine-learning model is trained by means of training data to generate a synthetic representation of an examination region of an examination object on the basis of at least one input representation of the examination region of the examination object.

The training data comprise a set of input data and target data for each examination object of a multiplicity of examination objects.

The term “multiplicity” means at least ten, preferably more than one hundred.

Each set of input data and target data includes at least one input representation of an examination region of the examination object as input data. Each set of input data and target data also includes a target representation of the examination region of the examination object and a transformed target representation as target data.

The examination region is normally the same for all examination objects.

Each input representation represents the examination region of the respective examination object in a first period of time before or after administration of a contrast agent.

Each target representation represents the examination region of the respective examination object in a second period of time after the administration of the contrast agent.

Each transformed target representation represents at least part of the examination region ofthe respective examination object in frequency space, if the target representation represents the examination region of the respective examination object in real space, or in real space, if the target representation represents the examination region of the respective examination object in frequency space.

In other words: if a target representation represents the examination region of the examination object in real space, the transformed target representation represents at least part of the examination region of the examination object in frequency space; if a target representation represents the examination region of the examination object in frequency space, the transformed target representation represents at least part of the examination region of the examination object in real space.

A transformed target representation in frequency space can be generated from a target representation in real space by Fourier transform, for example.

A transformed target representation in real space can be generated from a target representation in frequency space by inverse Fourier transform, for example.

The training of the machine-learning model involves feeding, for each examination object of the multiplicity of examination objects, the at least one input representation of the examination region to the machine-learning model. The model generates a synthetic representation on the basis of the at least one input representation of the examination region and on the basis of model parameters.

If the at least one input representation fed to the model represents the examination region in real space, the synthetic representation preferably (but not necessarily) also represents the examination region in real space.

If the at least one input representation fed to the model represents the examination region in frequency space, the synthetic representation preferably (but not necessarily) also represents the examination region in frequency space.

The machine-learning model may be configured and trained to generate on the basis of the at least one input representation of the examination region a synthetic representation of the examination region that represents i) the examination region in frequency space, if the at least one representation represents the examination region in real space, or ii) the examination region in real space, if the at least one representation represents the examination region in frequency space. In other words: the machine-learning model may be configured and trained to carry out (inter alia) a transform from real space into frequency space or vice versa.

Generated from or in relation to the synthetic representation is a transformed synthetic representation. If the synthetic representation represents the examination region in real space in a second period of time after administration of a contrast agent, the transformed synthetic representation represents at least part of the examination region in frequency space in the second period of time after the administration of the contrast agent. If the synthetic representation represents the examination region in frequency space in a second period of time after administration of a contrast agent, the transformed synthetic representation represents at least part of the examination region in real space in the second period of time after the administration of the contrast agent. The generation of the transformed synthetic representation can be achieved by transform of the synthetic representation and/or the machine-learning model may be configured and trained to generate a transformed synthetic representation on the basis of the at least one input representation.

A loss function is used to quantify the differences i) between at least part of the synthetic representation and at least part of the target representation and ii) between at least part of the transformed synthetic representation and at least part of the transformed target representation.

The loss function may comprise two terms: a first term for quantifying the differences between at least part of the synthetic representation and at least part of the target representation, and a second term for quantifying the differences between at least part of the transformed synthetic representation and at least part of the transformed target representation. The terms may, for example, be added up in the loss function. The terms may be weighted differently in the loss function. The following equation gives an example of a (total) loss function L for quantifying the differences:

L=λ₁·L₁+λ₂·L₂ (eq.1)

Here, L is the (total) loss function, L₁ is a term which represents the differences between the synthetic representation and the target representation, L₂ is a term which quantifies the differences between the transformed synthetic representation and the transformed target representation, and λ₁ and λ₂ are weight factors which, for example, can assume values between 0 and 1 and give a different weight to the two terms in the loss function. It is possible that the weight factors are kept constant or varied during the training of the machine-learning model.

Examples of loss functions that can be used to carry out the present invention are L1 loss function, L2 loss function, Lp loss function, structural similarity index measure (SSIM), VGG loss function, perceptual loss function or a combination of the aforementioned functions, or other loss functions. Further details on loss functions can be found, for example, in the scientific literature (see for example: R. Mechrez et al.: The Contextual Loss for Image Transformation with Non-Aligned Data, 2018, arXiv: 1803.02077v4; H. Zhao et al.: Loss Functions for Image Restoration with Neural Networks, 2018, arXiv:1511.08861v3; D. Fuoli et al.: Fourier Space Losses for Efficient Perceptual Image Super-Resolution, arXiv:2106.00783v1).

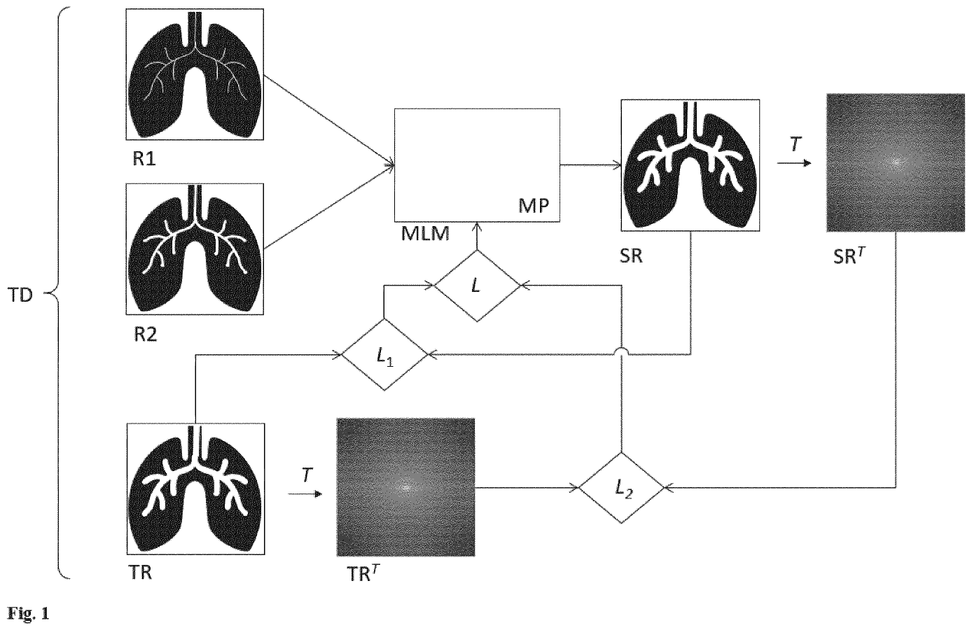

FIG. 1 shows by way of example and in schematic form the process of training the machine-learning model. The training is carried out with the aid of training data. FIG. 1 shows training data TD for an examination object. The training data TD comprise, as input data, a first input representation R1 of an examination region of the examination object and a second input representation R2 of the examination region of the examination object. The first input representation R1 and the second input representation R2 represent the examination region in real space. The first input representation R1 represents the examination region in a first period of time, for example at a first moment before administration of a contrast agent. The second input representation R2 also represents the examination region in the first period of time, for example at a second moment after the administration of a contrast agent.

The training data TD also comprise a target representation TR as target data. The target representation TR also represents the examination region in real space. The target representation TR represents the examination region in a second period of time, for example at a third moment after the administration of the contrast agent. The second period of time preferably follows the first period of time, i.e. the first moment is earlier than the second moment and the second moment is earlier than the third moment.

The machine-learning model MLM is trained to predict the target representation TR on the basis of the first input representation R1 and the second input representation R2 and on the basis of model parameters MP. The first input representation R1 and the second input representation R2 are fed to the machine-learning model as input data. The machine-learning model is configured to generate a synthetic representation SR.

In the example shown in FIG. 1, what is generated from the synthetic representation SR by means of a transform T (e.g. a Fourier transform) is a transformed synthetic representation SR^{T}. Analogously, what is generated from the target representation TR by means of the transform T is a transformed target representation TR^{T}. The transformed synthetic representation SR^{T} and the transformed target representation TR^{T} are frequency-space depictions of the examination region in the second period of time after the administration of the contrast agent.

A first loss function L₁ is used for quantifying the differences between the synthetic representation SR and the target representation TR. A second loss function L₂ is used for quantifying the differences between the transformed synthetic representation SR^{T} and the transformed target representation TR^{T}. The loss values calculated by means of the loss functions L₁ and L₂ are combined in a total loss function L (e.g. by addition with or without weighting) to give a total loss value.

Model parameters MP are modified with respect to reducing the total loss value. The total loss value can be reduced with the aid of an optimization method, for example with the aid of a gradient descent method.

The process is repeated for a multiplicity of examination objects until the total loss value has reached a predefined (desired) minimum and/or until the total loss value cannot be reduced further by modifying model parameters.

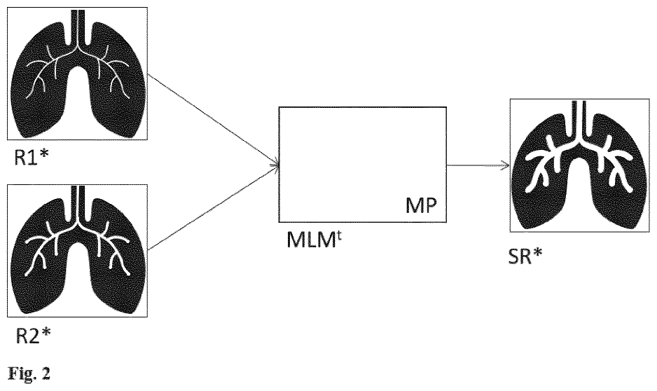

The trained machine-learning model can then be used for prediction. This is shown by way of example and in schematic form in FIG. 2: At least one input representation (R1*, R2*) of the examination region of a new examination object is fed to the trained machine-learning model MLM^{t}. The trained machine-learning model MLM^{t} generates a synthetic representation SR* of the examination region of the new examination object. Here, the term “new” means that input data from the new examination object has normally not already been used during the training and/or validation of the machine-learning model. The synthetic representation SR* generated may be output, stored and/or transmitted to a separate computer system.

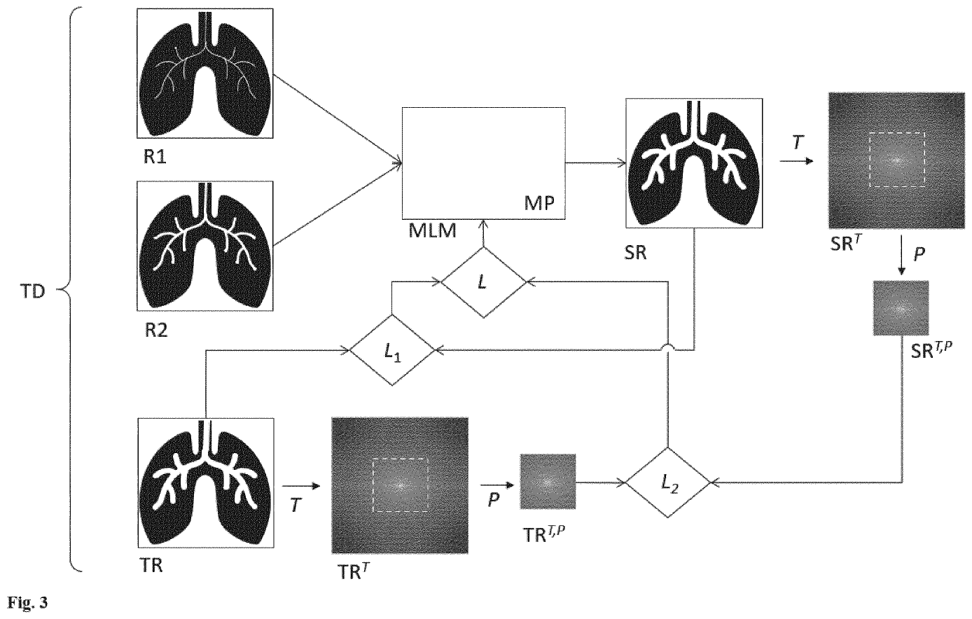

In one embodiment of the present disclosure, the quantification of the differences between the transformed synthetic representation and the transformed target representation is done only on the basis of portions of said representations. This is shown by way of example and in schematic form in FIG. 3.

The process shown in FIG. 3 corresponds to the process shown in FIG. 1, with the following differences:
The transformed target representation TR^{T} is reduced to a defined portion TR^{T,P}. The representation TR^{T,P} is part of the transformed target representation TR. The representation TR^{T,P} may be generated by a function P from the transformed target representation TR^{T}, for example by zeroing all values of frequencies outside the predefined part.
Analogously, the transformed synthetic representation SR^{T} is reduced to a defined portion SR^{T,P}.
The portions to which the transformed target representation TR^{T} and the transformed synthetic representation SR^{T} are reduced are normally mutually corresponding, i.e. they normally concern the same frequencies. In the process shown in FIG. 3, both the transformed target representation TR^{T} and the transformed synthetic representation SR^{T} have each been reduced to a portion containing the low frequencies in a region (e.g. rectangle or square) around the center (see the dashed white frames). Encoded in this region is mainly contrast information.

Therefore, in the example shown in FIG. 3, the total frequency space is not considered in the calculation by means of the loss function L₂. The representations in frequency space are reduced to a region of low frequencies; the higher frequencies are discarded. Encoded in the region of low frequencies is mainly contrast information, whereas encoded in the region of higher frequencies is mainly information about fine structures. This means that, in addition to generating the synthetic representation SR, the machine-learning model in the example shown in FIG. 3 is trained to correctly reproduce in particular the low frequencies. Thus the training focuses on contrast information.

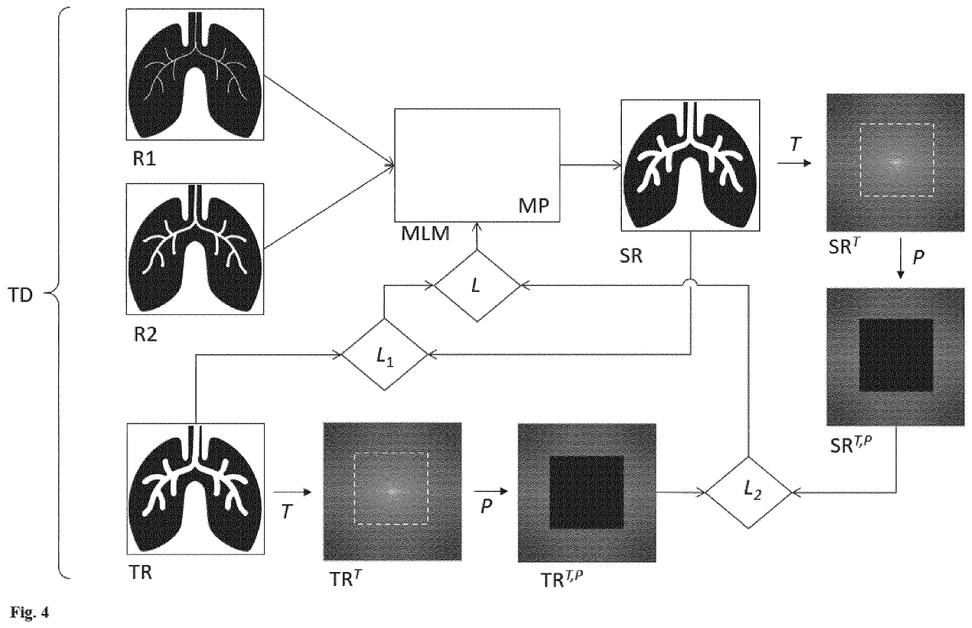

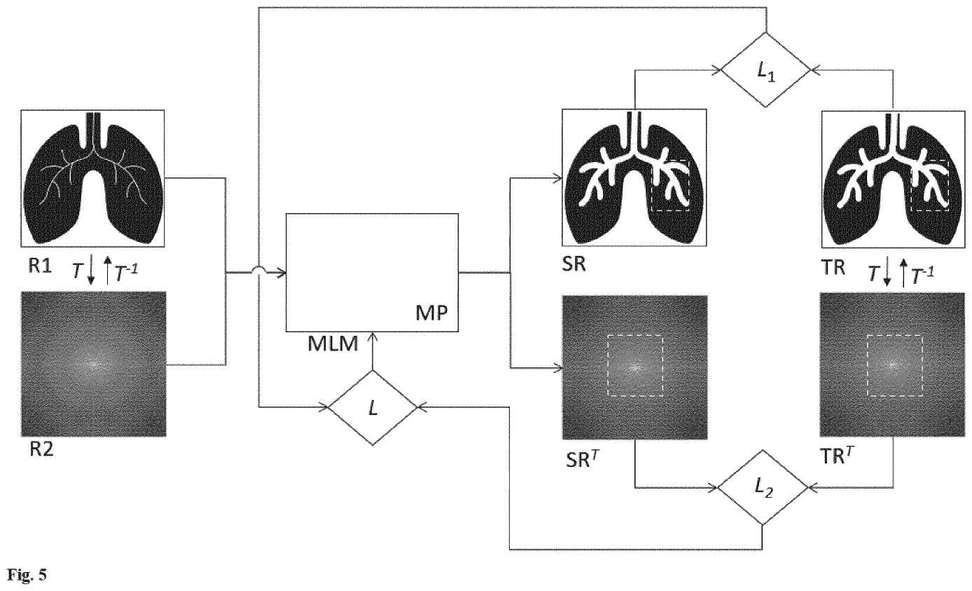

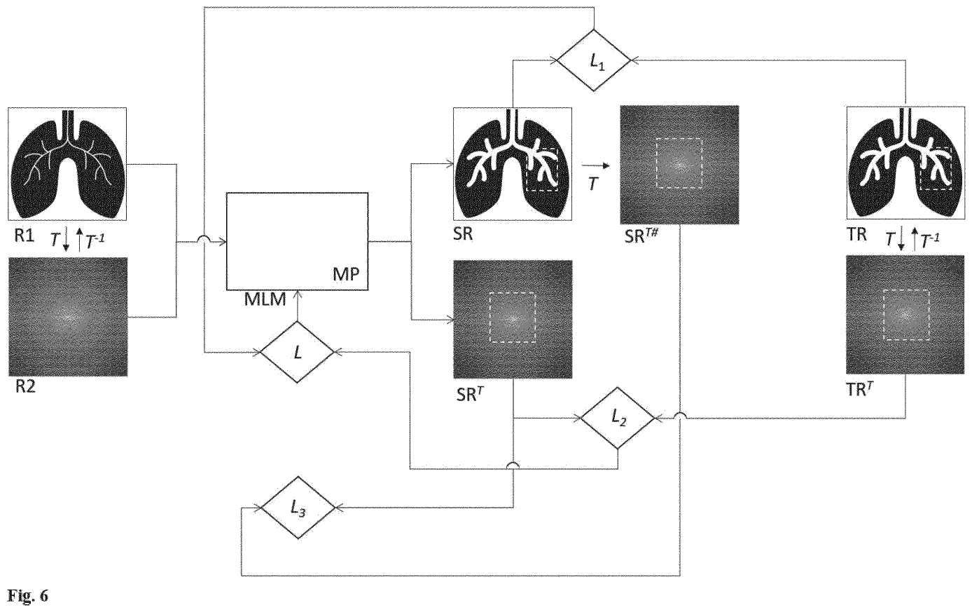

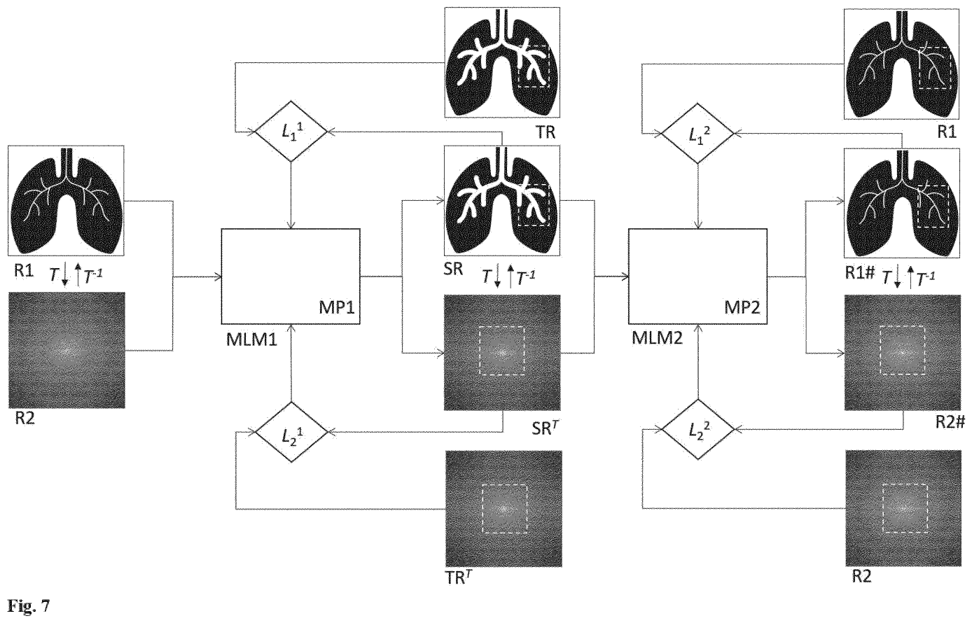

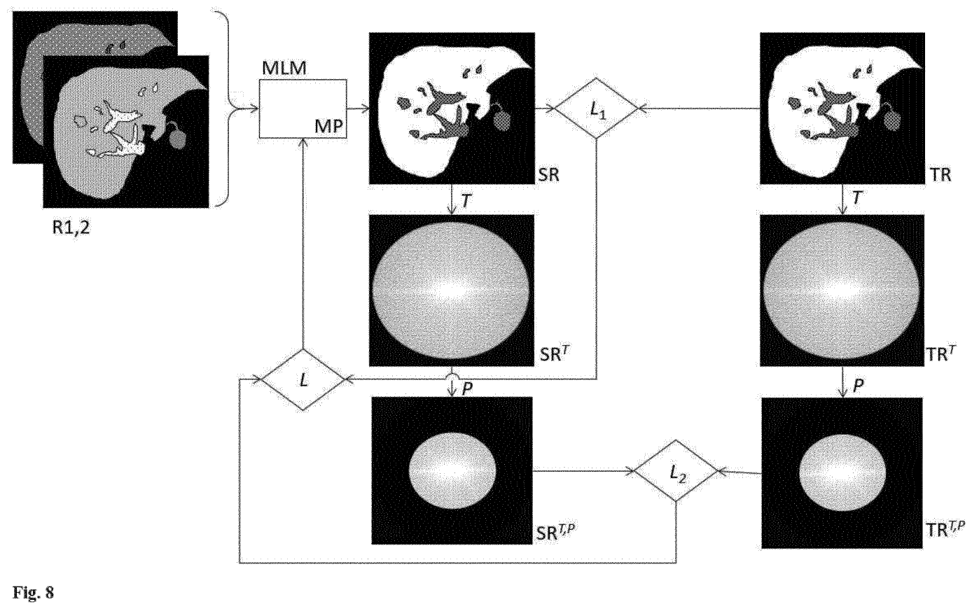

It should be noted that FIG. 3 is not to be understood as meaning that the transformed target representation TR^{T} and the transformed synthetic representation SR^{T} must be cropped to reduce them to the defined portion TR^{T,P} and the defined portion SR^{T,P}, respectively. It is also possible that the quantification of the differences between the portion SR^{T,P} and the portion TR^{T,P} by means of the loss function L₂ is achieved by only taking into account the regions SR^{T,P} and TR^{T,P} within the representations SR^{T} and TR^{T}. The term “reduce” is thus to be understood as meaning that the determination of the differences between the transformed synthetic representation SR^{T} and the transformed target representation TR^{T} is achieved by only taking into account the regions SR^{T,P} and TR^{T,P} within the representations SR^{T} and TR^{T}. This also applies analogously to FIG. 4, FIG. 5, FIG. 6, FIG. 7 and FIG. 8.

FIG. 4 shows another example in which the total frequency space is not considered and in which a defined frequency range is focused on instead. The process shown in FIG. 4 corresponds to the process shown in FIG. 3, with the difference that the function P ensures that the transformed target representation TR^{T} and the transformed synthetic representation SR^{T} are each reduced to a portion comprising higher frequencies, whereas low frequencies are discarded (the low frequency values are zeroed in the present example, the value of zero being represented by the color black). This means that, in addition to generating the synthetic representation SR, the machine-learning model in the example shown in FIG. 4 is trained to correctly reproduce in particular the higher frequencies and that the focus is thus on the correct reproduction of fine structures. As already explained in relation to FIG. 3, the low frequency values do not necessarily have to be zeroed; it is also possible that the quantification of the differences between the transformed synthetic representation SR^{T} and the transformed target representation TR^{T} by means of the loss function L₂ involves only taking into account the nonblack regions. This also applies analogously to FIG. 8.

It should also be noted that the part to which the frequency space is reduced for the loss calculation may also assume shapes and sizes other than those shown in FIG. 3, FIG. 4 and FIG. 8. Furthermore, multiple parts may be selected and the frequency-space depictions may be limited (reduced) to multiple parts (regions). For example, it is possible to divide the frequency space into various regions and to generate, for multiple regions or all the regions, frequency-space depictions of the examination region that each contain only the frequencies of the respective region. This makes it possible for different frequency ranges to be weighted differently/taken into account differently during training.

In the examples shown in FIG. 3 and FIG. 4, the representations R1, R2, TR and SR are representations in real space. It is also possible that one or more of these representations or all of them are frequency-space depictions.

If the transformed synthetic representation SR^{T} and the transformed target representation TR^{T} are representations in real space, a part (or multiple parts) in the representations may likewise be selected and the representations may be reduced to said part(s). In such a case, the loss calculation thus focuses on features in real space that are found in the selected part(s).

FIG. 5, FIG. 6, FIG. 7 and FIG. 8 show further embodiments of the training method.

In the embodiment shown in FIG. 5, the training data comprise for each examination object at least one input representation R1 of an examination region and a target representation TR of the examination region. The at least one input representation R1 represents the examination region in a first period of time before or after the administration of a contrast agent, and the target representation TR represents the examination region in a second period of time after the administration of the contrast agent.

The at least one input representation R1 and the target representation TR may each be a representation in real space or a representation in frequency space. In the example shown in FIG. 5, the input representation R1 and the target representation TR are representations in real space. The machine-learning model MLM is trained to predict the target representation TR on the basis of the at least one input representation R1. A transform T is used to generate a transformed input representation R2 on the basis of the input representation R1. Analogously, the transform T is used to generate a transformed target representation TR^{T} on the basis of the target representation TR. In the example shown in FIG. 5, the transformed input representation R2 and the transformed target representation TR^{T} are representations of the examination region in frequency space. The transformed input representation R2 represents the examination region (such as the input representation R1) in the first period of time; the transformed target representation TR^{T} represents the examination region (such as the target representation TR) in the second period of time.

FIG. 5 shows that the input representation R1 can be obtained by an inverse transform T¹ on the basis of the transformed input representation R2. Analogously, a target representation TR can be obtained by the inverse transform T¹ on the basis of the transformed target representation TR^{T}. The inverse transform T⁻¹ is the inverse transform in relation to the transform T, and this is indicated by the superscript index−1. The reference to the inverse transform is intended to make it clear that the training data must contain at least an input representation (R1 or R2) and a target representation (TR or TR^{T}); the other input representation (R2 or R1) and the other target representation (TR^{T} or TR) can be obtained from the existing representation by transform or inverse transform. This is generally applicable and does not only apply to the embodiment shown in FIG. 5.

In the embodiment shown in FIG. 5, both the input representation R1 and the transformed input representation R2 are fed to the machine-learning model MLM. The machine-learning model MLM is configured to generate both a synthetic representation SR and a transformed synthetic representation SR^{T}.

A first loss function L₁ is used to quantify the differences between at least part of the synthetic representation SR and at least part of the target representation TR. A second loss function L₂ is used to quantify the differences between at least part of the transformed synthetic representation SR^{T} and at least part of the transformed target representation TR^{T}. The dashed white frames in the transformed representations in FIG. 5 are intended to express that loss calculation by means of the loss function as described in relation to FIG. 3 and FIG. 4 does not have to be based on the entire frequency-space depiction and that, instead, the transformed representations can be reduced to a frequency range (or multiple frequency ranges). Analogously, the real-space depictions SR and TR can be reduced to one part (or multiple parts) for loss calculation (also shown by the dashed white frames). This also applies analogously to the other embodiments and does not only apply to the embodiment shown in FIG. 5.

The loss values calculated by means of the loss functions L₁ and L₂ are combined in a total loss function L (e.g. by addition with or without weighting) to give a total loss value.

Model parameters MP are modified with respect to reducing the total loss value. This can be done by means of an optimization method, for example a gradient descent method.

The process is repeated for a multiplicity of examination objects until the total loss value has reached a predefined (desired) minimum and/or until the total loss value cannot be reduced further by modifying model parameters.

The synthetic representation SR and the transformed synthetic representation SR^{T} must be interconvertible just like the target representation TR and the transformed target representation TR^{T}. It is possible to introduce a further loss function that assesses the quality of such a conversion. It is thus possible to generate a transformed synthetic representation SR^{T} from the synthetic representation SR by the transform T and to quantify the differences between this transformed synthetic representation generated by transform and the transformed synthetic representation generated by the machine-learning model by means of a third loss function L₃. Alternatively or additionally, it is possible to generate a synthetic representation SR from the transformed synthetic representation SR^{T} by the inverse transform T⁻¹ and to quantify the differences between this synthetic representation generated by inverse transform and the synthetic representation generated by the machine-learning model by means of a third loss function L₃. This is shown schematically in FIG. 6 using the example of generating a transformed synthetic representation SR^{T}* from the synthetic representation SR by transform T. In the example shown in FIG. 6, the loss function L₃ quantifies the differences between the transformed synthetic representation SR^{T#} generated by transform and the transformed synthetic representation SR^{T} generated by the machine-learning model MLM. The loss functions L₁, L₂ and L₃ are combined in a total loss function L (e.g. by addition with or without weighting). In the total loss function L, the individual terms may have different weights, and the weights may be kept constant or varied over the course of the training.

A modification of the embodiments shown in FIG. 5 and FIG. 6 is that the machine-learning model MLM is not fed the two input representations R1 and R2, but just one of the two representations. The machine-learning model may be configured and trained to generate the other of the two representations.

FIG. 7 shows in schematic form a further embodiment of the training method. In this embodiment, two machine-learning models are used: a first model MLM1 and a second model MLM2. The first machine-learning model MLM1 is trained to predict a target representation TR and/or a transformed target representation TR^{T} from an input representation R1 and/or R2. The second machine-learning model MLM2 is trained to predict the original input representation R1 and/or R2 again (to reconstruct it/them) from a predicted target representation SR and/or a predicted transformed target representation SR^{T}. The machine-learning models thus perform a cycle which can improve the quality of prediction by the first model MLM1 (which is used for later prediction).

The first machine-learning model MLM1 is fed at least one input representation R1 and/or at least one transformed input representation R2 as input data. The first model MLM1 is configured to generate a synthetic representation SR and/or a transformed synthetic representation SR^{T} on the basis of the input data and model parameters MP1. A transformed synthetic representation SR^{T} may also be generated by transform T of the synthetic representation SR. A synthetic representation SR may also be generated by inverse transform T⁻¹ of the transformed synthetic representation SR^{T}.

Differences between the synthetic representation SR and the target representation TR may be quantified by means of a loss function L₁¹. Differences between the transformed synthetic representation SR^{T} and the transformed target representation TR^{T} may be quantified by means of a loss function L₂¹.

Differences between a synthetic representation SR obtained by inverse transform T⁻¹ and the synthetic representation SR generated by the model MLM1 may be quantified by means of a loss function L₃¹ (not shown in FIG. 7). Alternatively or additionally, differences between a transformed synthetic representation SR^{T} obtained by transform T and the transformed synthetic representation SR^{T} generated by the model MLM1 may be quantified by means of a loss function L₄¹ (not shown in FIG. 6).

The loss functions L₁¹, L₂¹ and, if present, L₃¹ and/or L₄¹ may be combined to form a total loss function (not shown in FIG. 7).

Model parameters MP1 may be modified with respect to reducing the total loss value.

The synthetic representation SR and/or the transformed synthetic representation SR^{T} is/are fed to the second machine-learning model MLM2. The second model MLM2 is configured to reconstruct (predict) the first input representation R1 and/or the second input representation R2. The second model MLM2 is configured to generate a predicted first input representation R1 # and/or a predicted second input representation R2 # on the basis of the synthetic representation SR and/or the transformed synthetic representation SR^{T} and on the basis of model parameters MP2. A second input representation R2 # may also be generated by transform T of the first input representation R1 # and/or a first input representation R1 # may also be generated by inverse transform T^{|-1} of the second input representation R2 #.

Differences between the predicted first input representation R1 # and the first input representation R1 may be quantified by means of a loss function L₁². Differences between the predicted second input representation R2 # and the second input representation R2 may be quantified by means of a loss function L₂².

Differences between an input representation obtained by inverse transform T⁻¹ and the input representation generated by the model MLM2 may be quantified by means of a loss function L₃² (not shown in FIG. 7). Alternatively or additionally, differences between an input representation obtained by transform and the input representation generated by the model MLM2 may be quantified by means of a loss function L₄² (not shown in FIG. 7).

The loss functions L₁², L₂² and, if present, L₃² and/or L₄² may be combined to form a total loss function (not shown in FIG. 7).

Model parameters MP2 may be modified with respect to reducing the total loss value.

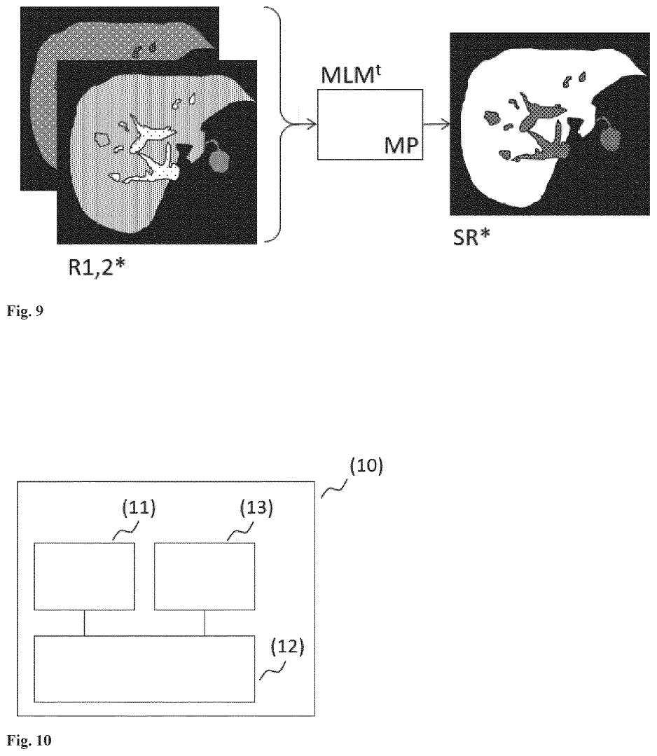

FIG. 8 shows in schematic form a further example of a method for training a machine-learning model. FIG. 9 shows the use of the trained machine-learning model for prediction. The example shown in FIG. 8 and FIG. 9 is concerned with quickening a magnetic resonance imaging examination of the liver of an examination object using a hepatobiliary contrast agent.

An example of a hepatobiliary contrast agent is the disodium salt of gadoxetic acid (Gd-EOB-DTPA disodium, GD), which is described in U.S. Pat. No. 6,039,931A and is commercially available under the trade names Primovist® and Eovist®.

A hepatobiliary contrast agent can be used for the detection of tumors in the liver. Blood is supplied to healthy liver tissue primarily via the portal vein (vena portae), whereas most primary tumors are supplied by the liver artery (arteria hepatica). After intravenous injection of a bolus of contrast agent, it is accordingly possible to observe a time delay between the signal increase in the healthy liver parenchyma and in the tumor.

Besides malignant tumors, what are commonly found in the liver are benign lesions such as cysts, hemangiomas and focal nodular hyperplasias (FNH). A proper planning of therapy requires that these be differentiated from the malignant tumors. Primovist® can be used for the identification of benign and malignant focal liver lesions. By means of T1-weighted MRI, it provides information about the character of said lesions. Differentiation is achieved by utilizing the difference in blood supply to liver and tumor and the time profile of contrast enhancement.

In the case of the contrast enhancement achieved by Primovist® during the wash-in phase, what are observed are typical perfusion patterns which provide information for the characterization of the lesions. Depicting the vascularization helps to characterize the lesion types and to determine the spatial relationship between tumor and blood vessels.

In the case of T1-weighted MRI images, Primovist® leads, 10-20 minutes after the injection (in the hepatobiliary phase), to a distinct signal enhancement in the healthy liver parenchyma, whereas lesions containing no hepatocytes or only a few hepatocytes, for example metastases or moderately to poorly differentiated hepatocellular carcinomas (HCCs), appear as darker regions.

Tracking the spread of the contrast agent over time thus provides a good way of detecting and differentially diagnosing focal liver lesions; however, the examination extends over a comparatively long period of time. Over this period of time, movements by the patient should be largely avoided in order to minimize movement artifacts in the MRI images. The lengthy restriction of movement can be unpleasant for a patient.

Accordingly, it is already proposed in WO2021/052896A1 that MRI images of the liver of a patient during the hepatobiliary phase not be generated by measurement, but instead be predicted on the basis of MRI images from one or more preceding phases.

FIG. 8 and FIG. 9 show an improved approach compared to the method described in WO2021/052896A1:

The machine-learning model MLM is trained on the basis of training data. The training data comprise, for each examination object of a multiplicity of examination objects, one or more input representations R1,2 of the liver or part of the liver during a first period of time before and/or after the administration of a hepatobiliary contrast agent and at least one target representation TR of the liver or part of the liver during a second period of time after the administration of the hepatobiliary contrast agent.

The input representation(s) R1,2 and the target representation TR are normally generated using an MRI scanner. The hepatobiliary contrast agent may, for example, be administered as a bolus into an arm vein of the examination object in a dose based on body weight.

The input representation(s) R1,2 may represent the liver or part of the liver during the native phase, arterial phase, portal venous phase and/or transitional phase (as described in WO2021/052896A1 and the references cited therein).

The target representation shows the liver or part of the liver during the hepatobiliary phase, i.e. for example 10 to 20 minutes after the administration of the hepatobiliary contrast agent.

The input representation(s) R1,2 and the target representation TR shown in FIG. 8 are representations of the liver or part of the liver in real space. When using multiple input representations R1,2, the input representations may represent the examination region in different states, namely at different moments in the native phase, arterial phase, portal venous phase and/or transitional phase. The target representation TR represents the examination region in a further state, namely at a moment in the hepatobiliary phase.

The input representations R1,2 (with or without prior preprocessing, which may include, for example, motion correction, coregistration and/or color space conversion) are passed to a machine-learning model MLM.

The machine-learning model is configured to generate a synthetic representation SR on the basis of the input representation(s) R1,2 and on the basis of model parameters MP.

What is generated from the synthetic representation SR by means of a transform T (e.g. a Fourier transform) is a transformed synthetic representation SR^{T}.

The transformed synthetic representation SR^{T} is, for example, a representation of the liver or part of the liver during the hepatobiliary phase in frequency space.

The transformed synthetic representation SR^{T} is reduced by means of a function P to a predefined portion, generating a partial transformed synthetic representation SR^{T,P}. The function P reduces the transformed synthetic representation SR^{T} to a predefined frequency range. In the present example, frequencies outside a circle of a defined radius around the origin of the transformed synthetic representation SR^{T} are zeroed, such that only the frequencies inside the circle remain. In other words, the higher frequencies in which fine-structure information is encoded are deleted, and what remain are the low frequencies in which contrast information is encoded.

An analogous procedure is used for the target representation TR:

What is generated from the target representation TR by means of a transform T (e.g. a Fourier transform) is a transformed target representation TR^{T}. The transformed target representation TR^{T} is a representation of the liver or part of the liver during the hepatobiliary phase in frequency space. The transformed target representation TR^{T} is reduced by means of the function P to a predefined portion, generating a partial transformed target representation TR^{TP}. The function P2 reduces the transformed target representation TR^{T} to the same frequency range as the transformed synthetic representation SR^{T}. In the present example, frequencies outside a circle of a defined radius around the origin of the transformed target representation TR^{T} are zeroed, such that only the frequencies inside the circle remain. In other words, the higher frequencies in which fine-structure information is encoded are deleted, and what remain are the low frequencies in which contrast information is encoded.

The quality of prediction by the machine-learning model is assessed by calculating a loss value by means of a loss function L. In the present example, the loss function L is composed of two terms: a first loss function L₁ and a second loss function L₂.

The first loss function L₁ quantifies the differences between the synthetic representation SR and the target representation TR.

The second loss function L₂ quantifies the differences between the partial transformed synthetic representation SR^{T} and the partial transformed target representation TR^{T}.

The terms for L₁ and L₂ may, for example, be provided with weight factors and added up in the total loss function L, as shown in the equation eq. 1 above.

In an optimization method, for example a gradient descent method, the model parameters MP can be modified with respect to reducing the loss value calculated by means of the total loss function L.

The process described is repeated for the other examination objects of the multiplicity of examination objects. The training can be ended when the loss values calculated by means of the total loss function L reach a defined minimum, i.e. the quality of prediction reaches a desired level.

FIG. 9 shows the use of the trained machine-learning model for prediction. The model may have been trained as described in relation to FIG. 8. The trained machine-learning model MLM^{t} is fed one or more input representations R1,2* of the liver or part of the liver of a new examination object. Here, the term “new” means that the input representations R1,2* have not already been used in training the model.

The input representation(s) R1,2* represent(s) the liver or part of the liver of the new examination object before and/or after the administration of a hepatobiliary contrast agent, which for example may have been administered in the form of a bolus into an arm vein of the new examination object.

The input representation(s) R1,2* represent(s) the liver or part of the liver of the new examination object in the native phase, arterial phase, portal venous phase and/or transitional phase.

The input representation(s) R1,2* represent(s) the liver or part of the liver ofthe new examination object in real space.

The trained machine-learning model is configured and trained to predict a synthetic representation SR* on the basis of the input representation(s) R1,2*.

The synthetic representation SR* represents the liver or part of the liver during the hepatobiliary phase, i.e. for example 10 to 20 minutes after the administration of the hepatobiliary contrast agent.

The machine-learning models according to the present disclosure may, for example, be an artificial neural network or include such an artificial neural network or a plurality thereof.

An artificial neural network comprises at least three layers of processing elements: a first layer with input neurons (nodes), an N-th layer with at least one output neuron (nodes), and N-2 inner layers, where N is a natural number and greater than 2.

The input neurons serve to receive the input representations. Normally, there is one input neuron for each pixel or voxel of an input representation when the representation is a real-space depiction in the form of a raster graphic, or one input neuron for each frequency present in the input representation when the representation is a frequency-space depiction. There may be additional input neurons for additional input values (for example information about the examination region, information about the examination object, information about the conditions when the input representation was generated, information about the state represented by the input representation, and/or information about the moment at which or period of time in which the input representation had been generated).

The output neurons may serve to output a synthetic representation representing the examination region in a different state.

The processing elements of the layers between the input neurons and the output neurons are connected to one another in a predetermined pattern with predetermined connection weights.

Preferably, the artificial neural network is a so-called convolutional neural network (CNN for short) or includes such a network.

A CNN normally consists essentially of an alternately repeating array of filters (convolutional layer) and aggregation layers (pooling layer) terminating in one or more layers of “normal” fully connected neurons (dense/fully connected layer).

The training of the neural network may, for example, be carried out by means of a back propagation method. The goal for the network is to map the input representation(s) onto the synthetic representation as reliably as possible. The quality of prediction is described by a loss function. The goal is to minimize the loss function. In the case of the back propagation method, an artificial neural network is taught by altering the connection weights.

In the trained state, the connection weights between the processing elements contain information regarding the dynamics of the relationship between the input representation(s) and the synthetic representation that can be used to predict, on the basis of one or more representations of the examination region of a new examination object, a synthetic representation of the examination region of the new examination object. Here, the term “new” means that representations of the examination region of the new examination object were not already used during the training of the machine-learning model.

A cross-validation method may be used to divide the data into training data sets and validation data sets. The training data set is used in the back propagation training of the network weights. The validation data set is used to check the accuracy of prediction with which the trained network can be applied to unknown (new) data.

The artificial neural network may have an autoencoder architecture; for example the artificial neural network may have an architecture such as the U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M.-Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The artificial neural network may be a regularized generative adversarial network (see for example Q. Li et al.: RegGAN: An End-to-End Network for Building Footprint Generation with Boundary Regularization, Remote Sens. 2022, 14, 1835).

The artificial neural network may be a conditional adversarial network (see for example P. Isola et al.: Image-to-Image Translation with Conditional Adversarial Networks, arXiv:1611.07004 [cs.CV]).

The artificial neural network may be a transformer network (see for example D. Karimi et al.: Convolution-Free Medical Image Segmentation using Transformers, arXiv:2102.13645 [eess.IV]).

FIG. 10 shows by way of example and in schematic form a computer system according to the present disclosure, which can be used for training the machine-learning model and/or for using the trained machine-learning model for prediction.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (10) shown in FIG. 10 comprises a receiving unit (11), a control and calculation unit (12) and an output unit (13).

The control and calculation unit (12) serves for control of the computer system (10), for coordination of the data flows between the units of the computer system (10), and for the performance of calculations.

The control and calculation unit (12) is configured:
to cause the receiving unit (11) to receive at least one input representation of an examination region of an examination object,
to input the received at least one input representation into a trained machine-learning model, the trained machine-learning model having been trained as described in this description,
to receive from the machine-learning model a synthetic representation of the examination region of the examination object,
to cause the output unit (13) to output the synthetic representation of the examination region of the examination object and/or to store it and/or to transmit it to a separate computer system.

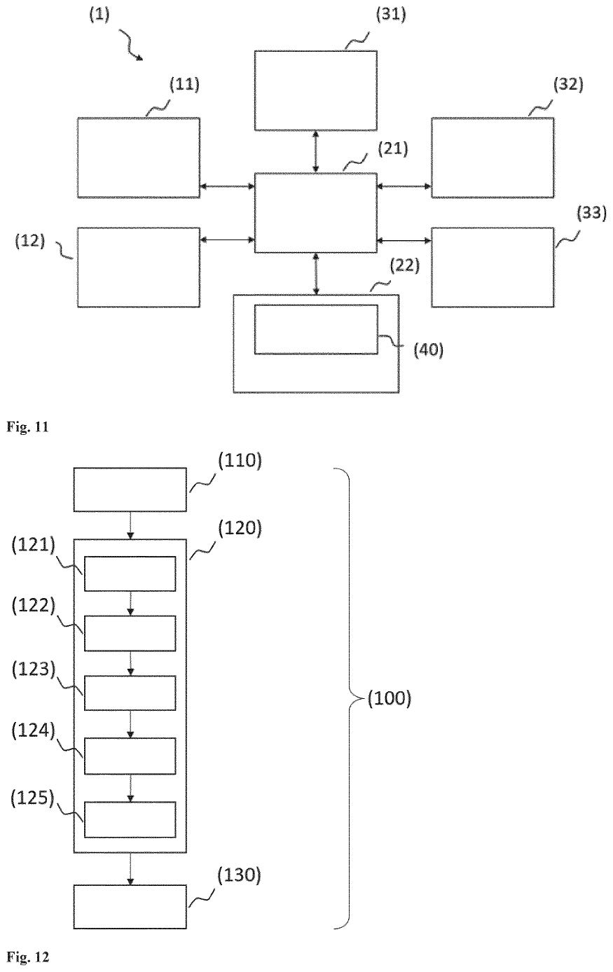

FIG. 11 shows by way of example and in schematic form a further embodiment of the computer system according to the invention.

The computer system (1) comprises a processing unit (21) connected to a memory (22). The processing unit (21) and the memory (22) form a control and calculation unit, as shown in FIG. 10.

The processing unit (21) may comprise one or more processors alone or in combination with one or more memories. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected notjustto the memory (22), but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as cellphone, Wi-Fi, satellite, cable, DSL, optical fiber and/or the like. In some examples, the one or more communication interfaces may comprise one or more close-range communication interfaces configured to connect devices with close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may furthermore include one or more interfaces for communication with peripherals such as printers and the like.

One or more computer programs (40) may be stored in the memory (22) and executed by the processing unit (21), which is thereby programmed to perform the functions described in this description. The retrieving, loading and execution of instructions of the computer program (40) may take place sequentially, such that an instruction is respectively retrieved, loaded and executed. However, the retrieving, loading and/or execution may also take place in parallel.

The machine-learning model according to the invention may also be stored in the memory (22).

The computer system according to the invention may be designed as a laptop, notebook, netbook and/or tablet PC; it may also be a component of an MRI scanner, a CT scanner or an ultrasound diagnostic device.

FIG. 12 shows schematically in the form of a flow chart one embodiment of the method for training a machine-learning model. The training method (100) comprises the steps of
(110) receiving and/or providing training data, the training data comprising a set of input data and target data for each examination object of a multiplicity of examination objects,
   each set comprising at least one input representation of an examination region of the examination object as input data and a target representation of the examination region of the examination object and a transformed target representation as target data,
   the at least one input representation representing the examination region in a first period of time before and/or after the administration of a contrast agent and the synthetic representation representing the examination region in a second period of time after the administration of the contrast agent,
   the transformed target representation representing at least part of the examination region of the examination object
      in frequency space, if the target representation represents the examination region of the examination object in real space, or
      in real space, if the target representation represents the examination region of the examination object in frequency space,
(120) training a machine-learning model, the machine-learning model being configured to generate on the basis of at least one input representation of an examination region of an examination object and model parameters a synthetic representation of the examination region of the examination object,
   wherein the training comprises for each examination object of the multiplicity of examination objects:
      (121) feeding the at least one input representation to the machine-learning model,
      (122) receiving a synthetic representation of the examination region of the examination object from the machine-learning model,
      (123) generating and/or receiving a transformed synthetic representation on the basis of the synthetic representation and/or in relation to the synthetic representation, the transformed synthetic representation representing at least part of the examination region of the examination object
         in frequency space, if the synthetic representation represents the examination region of the examination object in real space, or
         in real space, if the synthetic representation represents the examination region of the examination object in frequency space,
      (124) quantifying the differences i) between at least part of the synthetic representation and at least part of the target representation and ii) between at least part of the transformed synthetic representation and at least part of the transformed target representation by means of a loss function,
      (125) reducing the differences by modifying model parameters,
(130) outputting and/or storing the trained machine-learning model and/or the model parameters and/or transmitting the trained machine-learning model and/or the model parameters to a separate computer system and/or using the trained machine-learning model for generation of a synthetic radiological image of the examination region of a new examination object.

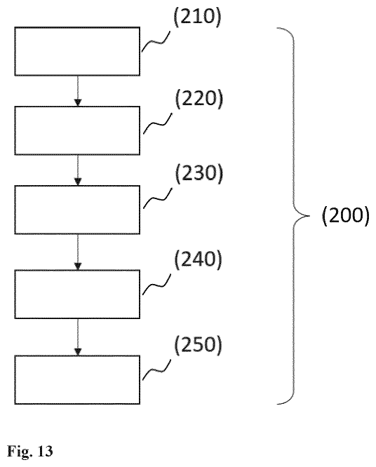

FIG. 13 shows schematically in the form of a flow chart one embodiment of the method for generating a synthetic representation of an examination region of an examination object with the aid of the trained machine-learning model. The prediction method (200) comprises the steps of
(210) providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) at least one input representation of the examination region of the examination object, ii) a target representation of the examination region of the examination object and iii) a transformed target representation,
      the at least one input representation representing the examination region of the respective examination object in a first period of time before or after administration of a contrast agent,
      the target representation representing the examination region of the respective examination object in a second period of time after the administration of the contrast agent,
      the transformed target representation representing at least part of the examination region of the respective examination object in frequency space, if the target representation represents the examination region of the respective examination object in real space, or in real space, if the target representation represents the examination region of the respective examination object in frequency space,
   the training of the machine-learning model comprising reducing differences between i) at least part of the synthetic representation and at least part of the target representation and ii) between at least part of a transformed synthetic representation and at least part of the transformed target representation,
(220) receiving at least one input representation of the examination region of a new examination object, the at least one input representation of the examination region of the new examination object representing the examination region in a first period of time before and/or after administration of a contrast agent,
(230) inputting the at least one input representation of the examination region of the new examination object into the trained machine-learning model,
(240) receiving a synthetic representation of the examination region of the new examination object from the machine-learning model,
(250) outputting and/or storing the synthetic representation of the examination region of the new examination object and/or transmitting the synthetic representation of the examination region of the new examination object to a separate computer system.

1. A computer-implemented method comprising:
providing a trained machine-learning model (MLM^{t});
the trained machine-learning model (MLM^{t}) having been trained by means of training data (TD) to generate on the basis of at least one input representation (R1, R2) of an examination region of an examination object a synthetic representation (SR) of the examination region of the examination object; and
the training data (TD) comprising for each examination object of a multiplicity of examination objects i) at least one input representation (R1, R2) of the examination region of the examination object, ii) a target representation (TR) of the examination region of the examination object and iii) a transformed target representation (TR^{T});
the at least one input representation (R1, R2) representing the examination region of the respective examination object in a first period of time before or after administration of a contrast agent;
the target representation (TR) representing the examination region of the respective examination object in a second period of time after the administration of the contrast agent; and
the transformed target representation (TR^{T}) representing at least part of the examination region of the respective examination object in frequency space, if the target representation (TR) represents the examination region of the respective examination object in real space, or in real space, if the target representation (TR) represents the examination region of the respective examination object in frequency space;
the training of the machine-learning model (MLM^{t}) comprising reducing differences between i) at least part of the synthetic representation (SR) and at least part of the target representation (TR) and ii) between at least part of a transformed synthetic representation (SR^{T}) and at least part of the transformed target representation (TR^{T});
receiving at least one input representation (R1*, R2*) of the examination region of a new examination object, the at least one input representation (R1*, R2*) of the examination region of the new examination object representing the examination region in a first period of time before and/or after administration of the contrast agent;
inputting the at least one input representation (R1*, R2*) of the examination region of the new examination object into the trained machine-learning model (MLM^{t});
receiving a synthetic representation (SR*) of the examination region of the new examination object from the machine-learning model (MLM^{t}); and
outputting and/or storing the synthetic representation (SR*) of the examination region of the new examination object and/or transmitting the synthetic representation (SR*) of the examination region of the new examination object to a separate computer system.

2. The method as claimed in claim 1, wherein the training of the machine-learning model (MLM^{t}) comprises:
receiving and/or providing the training data (TD), the training data (TD) comprising a set of input data and target data for each examination object of the multiplicity of examination objects:
each set comprising the at least one input representation (R1, R2) of the examination region of the examination object as input data and the target representation (TR) of the examination region of the examination object and the transformed target representation (TR^{T}) as target data;
the at least one input representation (R1, R2) representing the examination region in the first period of time before and/or after the administration of the contrast agent and the target representation (TR) representing the examination region in the second period of time after the administration of the contrast agent; and
the transformed target representation (TR^{T}) representing at least part of the examination region of the examination object;
in frequency space, if the target representation (TR) represents the examination region of the examination object in real space; or
in real space, if the target representation (TR) represents the examination region of the examination object in frequency space;
training a machine-learning model (MLM), the machine-learning model (MLM) being configured to generate on the basis of at least one input representation (R1, R2) of an examination region of an examination object and model parameters (MP) a synthetic representation (SR) of the examination region of the examination object; and
wherein the training comprises for each examination object of the multiplicity of examination objects:
feeding the at least one input representation (R1, R2) to the machine-learning model;
receiving a synthetic representation (SR) of the examination region of the examination object from the machine-learning model (MLM); and
generating and/or receiving a transformed synthetic representation (SR^{T}) on the basis of the synthetic representation (SR) and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (TR) and ii) between at least part of the transformed synthetic representation (SR^{T}) and at least part of the transformed target representation (TR^{T}) by means of a loss function (L); and
reducing the differences by modifying model parameters (MP);
outputting and/or storing the trained machine-learning model (MLM^{t}) and/or the model parameters (MP) and/or transmitting the trained machine-learning model (MLM^{t}) and/or the model parameters (MP) to a separate computer system.

3. The method as claimed in claim 2,
wherein the receiving and/or providing of training data (TD) comprises:
generating a partial transformed target representation (TR^{T,P}), the partial transformed target representation (TR^{T,P}) being reduced to one part or multiple parts of the transformed target representation (TR^{T});
wherein the generating and/or receiving of a transformed synthetic representation (SR^{T}) on the basis of and/or in relation to the synthetic representation (SR) comprises:
generating a partial transformed synthetic representation (SR^{T,P}), the partial transformed synthetic representation (SR^{T,P}) being reduced to one part or multiple parts of the transformed synthetic representation (SR^{T}); and
wherein the quantifying of the differences between the transformed synthetic representation (SR^{T}) and the transformed target representation (TR^{T}) comprises:
quantifying the differences between the partial transformed synthetic representation (SR^{T,P}) and the partial transformed target representation (TR^{T,P}).

4. The method as claimed in claim 2,
wherein the training comprises for each examination object of the multiplicity of examination objects:
feeding the at least one input representation (R1, R2) to the machine-learning model (MLM) ;
receiving the synthetic representation (SR) and a first transformed synthetic representation (SR^{T}) of the examination region of the examination object from the machine-learning model (MLM), the first transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
generating a second transformed synthetic representation (SR^{T#}) on the basis of the synthetic representation (SR) by means of a transform (7), the second transformed synthetic representation (SR^{T#}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (TR), ii) between at least part of the first transformed synthetic representation (SR^{T}) and at least part of the transformed target representation (TR^{T}) and iii) between at least part of the first transformed synthetic representation (SR^{T}) and at least part of the second transformed synthetic representation (SR^{T#}) by means of a loss function (L); and
reducing the differences by modifying model parameters (MP).

5. The method as claimed in claim 1,
wherein the machine-learning model (MLM) undergoing training comprises a first machine-learning model (MLM1) and a second machine-learning model (MLM2);
wherein the first machine-learning model (MLM1) is configured to generate on the basis of at least one input representation (R1, R2) and model parameters (MP1) a synthetic representation (SR) of the examination region of the examination object;
wherein the second machine-learning model (MLM2) is configured to reconstruct on the basis of the synthetic representation (SR) of the examination region of the examination object and model parameters (MP2) at least one input representation (R1, R2);
wherein the training comprises for each examination object of the multiplicity of examination objects:
generating a transformed input representation (R2) on the basis of at least one input representation (R1) by means of a transform (7), the transformed input representation (R2) representing at least part of the examination region of the examination object;
in frequency space, if the at least one input representation (R1) represents the examination region of the examination object in real space; or
in real space, if the input representation (R1) represents the examination region of the examination object in frequency space;
feeding the at least one input representation (R1) and/or the transformed input representation (R2) to the first machine-learning model (MLM1) ;
receiving a synthetic representation (SR) of the examination region of the examination object from the first machine-learning model (MLM1); and
generating and/or receiving a transformed synthetic representation (SR) on the basis of and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
feeding the synthetic representation (SR) and/or the transformed synthetic representation (SR^{T}) to the second machine-learning model (MLM2);
receiving a predicted input representation (R1 #) from the second machine-learning model (MLM2);
generating and/or receiving a transformed predicted input representation (R2 #) on the basis of and/or in relation to the predicted input representation (R1 #), the transformed predicted input representation (R2 #) representing at least part of the examination region of the examination object;
in frequency space, if the predicted input representation (R1 #) represents the examination region of the examination object in real space; or
in real space, if the predicted input representation (R1 #) represents the examination region of the examination object in frequency space;
quantifying the differences i) between at least part of the synthetic representation (SR) and at least part of the target representation (TR), ii) between at least part of the transformed synthetic representation (SR^{T}) and at least part of the transformed target representation (TR^{T}), iii) between at least part of the input representation (R1) and at least part of the predicted input representation (R1 #) and iv) between at least part of the transformed input representation (R2) and at least part of the transformed predicted input representation (R2 #) by means of a loss function (L); and
reducing the differences by modifying model parameters.

6. The method as claimed in claim 1, wherein the examination object is a human.

7. The method as claimed in claim 1, wherein the examination region includes a liver, brain, heart, kidney, lung, stomach, intestine, pancreas, thyroid gland, prostate or breast of a human.

8. The method as claimed in claim 1, wherein the examination region is a liver or part of a liver of a human.

9. The method as claimed in claim 1, wherein each input representation of the at least one input representation (R1, R2, R1*, R2*) is a representation of the examination region in real space, the target representation (TR) is a representation of the examination region in real space, and the synthetic representation (SR, SR*) is a representation of the examination region in real space.

10. The method as claimed in claim 3, wherein the partial transformed synthetic representation (SR^{T,P}) represents the examination region in frequency space, the partial transformed synthetic representation (SR^{T,P}) being reduced to a frequency range of the transformed synthetic representation (SR^{T}), contrast information being encoded in the frequency range.

11. The method as claimed in claim 3, wherein the partial transformed synthetic representation (SR^{T,P}) represents the examination region in frequency space, the partial transformed synthetic representation (SR^{T,P}) being reduced to a frequency range of the transformed synthetic representation (SR^{T}), information about fine structures being encoded in the frequency range.

12. A computer system comprising
a receiving unit ;
a control and calculation unit ; and
an output unit;
wherein the control and calculation unit is configured to provide a trained machine-learning model (MLM^{t});
the trained machine-learning model (MLM) having been trained by means of training data (TD) to generate on the basis of at least one input representation (R1, R2) of an examination region of an examination object a synthetic representation (SR) of the examination region of the examination object;
the training data (TD) comprising for each examination object of a multiplicity of examination objects i) at least one input representation (R1, R2) of the examination region of the examination object, ii) a target representation (TR) of the examination region of the examination object and iii) a transformed target representation (TR^{T});
the at least one input representation (R1, R2) representing the examination region of the respective examination object in a first period of time before or after administration of a contrast agent;
the target representation (TR) representing the examination region of the respective examination object in a second period of time after the administration of the contrast agent; and
the transformed target representation (TR^{T}) representing at least part of the examination region of the respective examination object in frequency space, if the target representation (TR) represents the examination region of the respective examination object in real space, or in real space, if the target representation (TR) represents the examination region of the respective examination object in frequency space;
the training of the machine-learning model (MLM^{t}) comprising reducing differences between i) at least part of the synthetic representation (SR) and at least part of the target representation (TR) and ii) between at least part of a transformed synthetic representation (SR^{T}) and at least part of the transformed target representation (TR^{T});
wherein the control and calculation unit is configured to cause the receiving unit to receive at least one input representation (R1*, R2*) of an examination region of a new examination object, the at least one input representation (R1*, R2*) of the examination region of the new examination object representing the examination region in a first period of time before and/or after administration of the contrast agent;
wherein the control and calculation unit is configured to input the at least one input representation (R1*, R2*) of the examination region of the new examination object into a trained machine-learning model (MLM^{t});
wherein the control and calculation unit is configured to receive from the machine-learning model (MLM^{t}) a synthetic representation (SR*) of the examination region of the new examination object; and
wherein the control and calculation unit is configured to cause the output unit to output the synthetic representation (SR*) of the examination region of the new examination object and/or to store it and/or to transmit it to a separate computer system.

13. A computer program product comprising a computer program that can be loaded into a working memory of a computer system, wherein it causes the computer system to execute the following:
providing a trained machine-learning model (MLM^{t});
the trained machine-learning model (MLM^{t}) having been trained by means of training data (TD) to generate on the basis of at least one input representation (R1, R2) of an examination region of an examination object a synthetic representation (SR) of the examination region of the examination object;
the training data (TD) comprising for each examination object of a multiplicity of examination objects i) at least one input representation (R1, R2) of the examination region of the examination object, ii) a target representation (TR) of the examination region of the examination object and iii) a transformed target representation (TR^{T});
the at least one input representation (R1, R2) representing the examination region of the respective examination object in a first period of time before or after administration of a contrast agent;
the target representation (TR) representing the examination region of the respective examination object in a second period of time after the administration of the contrast agent;
the transformed target representation (TR^{T}) representing at least part of the examination region of the respective examination object in frequency space, if the target representation (TR) represents the examination region of the respective examination object in real space, or in real space, if the target representation (TR) represents the examination region of the respective examination object in frequency space;
the training of the machine-learning model (MLM^{t}) comprising reducing differences between i) at least part of the synthetic representation (SR) and at least part of the target representation (TR) and ii) between at least part of a transformed synthetic representation (SR^{T}) and at least part of the transformed target representation (TR^{T}); and
receiving at least one input representation (R1*, R2*) of the examination region of a new examination object, the at least one input representation (R1*, R2*) of the examination region of the new examination object representing the examination region in a first period of time before and/or after administration of a contrast agent;
inputting the at least one input representation (R1*, R2*) of the examination region of the new examination object into the trained machine-learning model (MLM^{t});
receiving a synthetic representation (SR*) of the examination region of the new examination object from the machine-learning model (MLM^{t}); and
outputting and/or storing the synthetic representation (SR*) of the examination region of the new examination object and/or transmitting the synthetic representation (SR*) of the examination region of the new examination object to a separate computer system.

14-16. (canceled)

17. The method as claimed in claim 1, wherein the contrast agent comprises one or more contrast agents selected from the following list:
gadoxetate disodium;
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(+)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of the formula (I) 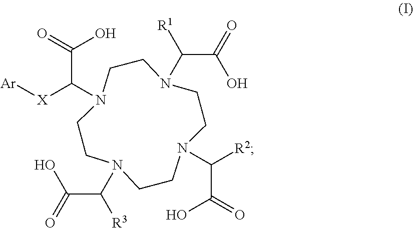
wherein
Ar is a group selected from 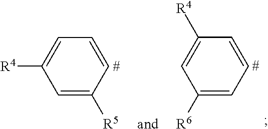
wherein _{#} is the linkage to X;
X is a group selected from
CH₂, (CH₂)₂, (CH₂)₃, (CH₂)₄ and *—(CH₂)₂O—CH₂— ^{#};
wherein * is the linkage to Ar and ^{#} is the linkage to the acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—
R⁵ is a hydrogen atom;
and
R⁶ is a hydrogen atom;
or a stereoisomer, tautomer, hydrate, solvate or salt thereof, or a mixture thereof;
a Gd³⁺ complex of a compound of the formula (II) 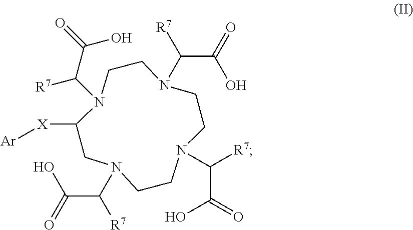
wherein
Ar is a group selected from 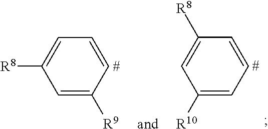
wherein is the linkage to X;
X is a group selected from CH₂, (CH₂)₂, (CH₂)₃, (CH₂)₄ and *—(CH₂)₂O—CH₂—^{#}, wherein * is the linkage to Ar and ^{#} is the linkage to the acetic acid residue;
R⁷ is a hydrogen atom or a group selected from C₁-C₃ alkyl, —CH₂OH;
(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

18. The computer system as claimed in claim 12, wherein the contrast agent comprises one or more contrast agents selected from the following list:
gadoxetate disodium;
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(+)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of the formula (I) 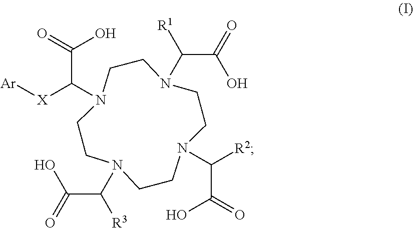
wherein
Ar is a group selected from 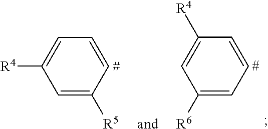
wherein _{#} is the linkage to X;
X is a group selected from
CH₂, (CH₂)₂, (CH₂)₃, (CH₂)₄ and *—(CH₂)₂O—CH₂—;
wherein * is the linkage to Ar and ^{#} is the linkage to the acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, tautomer, hydrate, solvate or salt thereof, or a mixture thereof,
a Gd³⁺ complex of a compound of the formula (II) 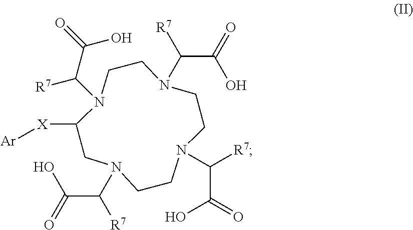
wherein
Ar is a group selected from 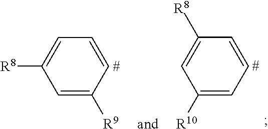
wherein _{#} is the linkage to X;
X is a group selected from CH₂, (CH₂)₂, (CH₂)₃, (CH₂)₄ and *—(CH₂)₂O—CH₂—^{#}, wherein * is the linkage to Ar and ^{#} is the linkage to the acetic acid residue;
R⁷ is a hydrogen atom or a group selected from C₁-C₃ alkyl, —CH₂OH;
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

19. The computer program product as claimed in claim 13, wherein the contrast agent comprises one or more contrast agents selected from the following list:
gadoxetate disodium;
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(+)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of the formula (I) 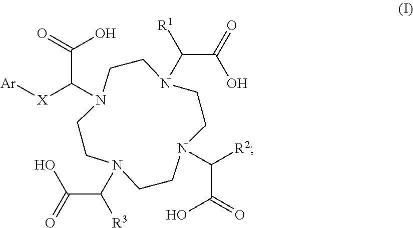
wherein
Ar is a group selected from 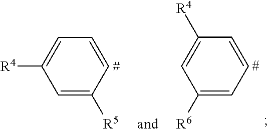
wherein _{#} is the linkage to X;
X is a group selected from
CH₂, (CH₂)₂, (CH₂)₃, (CH₂)₄ and *—(CH₂)₂O—CH₂;
wherein * is the linkage to Ar and ^{#} is the linkage to the acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, tautomer, hydrate, solvate or salt thereof, or a mixture thereof,
a Gd³⁺ complex of a compound of the formula (II) 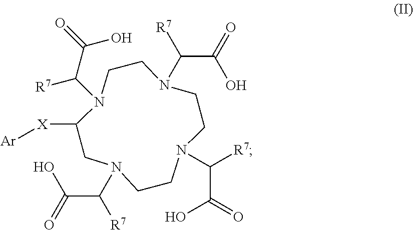
wherein
Ar is a group selected from 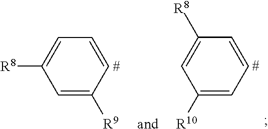
wherein _{#} is the linkage to X;
X is a group selected from CH₂, (CH₂)₂, (CH₂)₃, (CH₂)₄ and *—(CH₂)₂O—CH₂—, wherein * is the linkage to Ar and ^{#} is the linkage to the acetic acid residue;
R⁷ is a hydrogen atom or a group selected from C₁-C₃ alkyl, —CH₂OH;
—(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

20. The computer program product as claimed in claim 13,
wherein the training of the machine-learning model (MLM^{t}) comprises:
receiving and/or providing the training data (TD), the training data (TD) comprising a set of input data and target data for each examination object of the multiplicity of examination objects;
each set comprising the at least one input representation (R1, R2) of the examination region of the examination object as input data and the target representation (TR) of the examination region of the examination object and the transformed target representation (TR^{T}) as target data;
the at least one input representation (R1, R2) representing the examination region in the first period of time before and/or after the administration of the contrast agent and the target representation (TR) representing the examination region in the second period of time after the administration of the contrast agent; and
the transformed target representation (TR^{T}) representing at least part of the examination region of the examination object;
in frequency space, if the target representation (TR) represents the examination region of the examination object in real space; or
in real space, if the target representation (TR) represents the examination region of the examination object in frequency space;
training a machine-learning model (MLM), the machine-learning model (MLM) being configured to generate on the basis of at least one input representation (R1, R2) of an examination region of an examination object and model parameters (MP) a synthetic representation (SR) of the examination region of the examination object; and
wherein the training comprises for each examination object of the multiplicity of examination objects:
feeding the at least one input representation (R1, R2) to the machine-learning model;
receiving a synthetic representation (SR) of the examination region of the examination object from the machine-learning model (MLM); and
generating and/or receiving a transformed synthetic representation (SR) on the basis of the synthetic representation (SR) and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (TR) and ii) between at least part of the transformed synthetic representation (SR^{T}) and at least part of the transformed target representation (TR^{T}) by means of a loss function (L); and
reducing the differences by modifying model parameters (MP);
outputting and/or storing the trained machine-learning model (MLM^{t}) and/or the model parameters (MP) and/or transmitting the trained machine-learning model (MLM^{t}) and/or the model parameters (MP) to a separate computer system.

21. The computer program product as claimed in claim 13, wherein the training comprises for each examination object of the multiplicity of examination objects:
feeding the at least one input representation (R1, R2) to the machine-learning model (MLM);
receiving the synthetic representation (SR) and a first transformed synthetic representation (SR^{T}) of the examination region of the examination object from the machine-learning model (MLM), the first transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
generating a second transformed synthetic representation (SR^{T#}) on the basis of the synthetic representation (SR) by means of a transform (7), the second transformed synthetic representation (SR^{T#}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (TR), ii) between at least part of the first transformed synthetic representation (SR^{T}) and at least part of the transformed target representation (TR^{T}) and iii) between at least part of the first transformed synthetic representation (SR^{T}) and at least part of the second transformed synthetic representation (SR^{T#}) by means of a loss function (L); and
reducing the differences by modifying model parameters (MP).

22. The computer program product as claimed in claim 13, wherein the machine-learning model (MLM) undergoing training comprises a first machine-learning model (MLM1) and a second machine-learning model (MLM2);
wherein the first machine-learning model (MLM1) is configured to generate on the basis of at least one input representation (R1, R2) and model parameters (MP1) a synthetic representation (SR) of the examination region of the examination object;
wherein the second machine-learning model (MLM2) is configured to reconstruct on the basis of the synthetic representation (SR) of the examination region of the examination object and model parameters (MP2) at least one input representation (R1, R2);
wherein the training comprises for each examination object of the multiplicity of examination objects:
generating a transformed input representation (R2) on the basis of at least one input representation (R1) by means of a transform (7), the transformed input representation (R2) representing at least part of the examination region of the examination object;
in frequency space, if the at least one input representation (R1) represents the examination region of the examination object in real space; or
in real space, if the input representation (R1) represents the examination region of the examination object in frequency space;
feeding the at least one input representation (R1) and/or the transformed input representation (R2) to the first machine-learning model (MLM1);
receiving a synthetic representation (SR) of the examination region of the examination object from the first machine-learning model (MLM1); and
generating and/or receiving a transformed synthetic representation (SR^{T}) on the basis of and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
feeding the synthetic representation (SR) and/or the transformed synthetic representation (SR^{T}) to the second machine-learning model (MLM2);
receiving a predicted input representation (R1 #) from the second machine-learning model (MLM2);
generating and/or receiving a transformed predicted input representation (R2 #) on the basis of and/or in relation to the predicted input representation (R1 #), the transformed predicted input representation (R2 #) representing at least part of the examination region of the examination object;
in frequency space, if the predicted input representation (R1 #) represents the examination region of the examination object in real space; or
in real space, if the predicted input representation (R1 #) represents the examination region of the examination object in frequency space;
quantifying the differences i) between at least part of the synthetic representation (SR) and at least part of the target representation (TR), ii) between at least part of the transformed synthetic representation (SR^{T}) and at least part of the transformed target representation (TR^{T}), iii) between at least part of the input representation (R1) and at least part of the predicted input representation (R1 #) and iv) between at least part of the transformed input representation (R2) and at least part of the transformed predicted input representation (R2 #) by means of a loss function (L); and
reducing the differences by modifying model parameters.

23. The computer program product as claimed in claim 13, wherein the examination object is a human.
